(19) United States
(12) Patent Application Publication
(10) Pub. No.: US 2024/0285737 A1
(43) Pub. Date: Aug. 29, 2024

(54) IMPROVED METHODS OF TREATMENT USING IMMUNOGENIC PEPTIDES

(71) Applicant: IMCYSE SA, Angleur (Liège) (BE)

(72) Inventor: Jean VAN RAMPELBERGH, Bruxelles (BE)

(21) Appl. No.: 18/564,985

(22) PCT Filed: Jun. 1, 2022
(86) PCT No.: PCT/EP2022/064848

§ 371 Date: Nov. 28, 2023

(30) Foreign Application Priority Data
Jun. 1, 2021 (EP) 21177145.6

(51) Int. Cl. A61K 39/00; A61P 37/06

(52) U.S. Cl. CPC A61K 39/0008; A61K 39/001114; A61P 37/06; A61K 2039/572

(57) ABSTRACT

The present invention relates to a specific administration scheme of an immunogenic peptide comprising T cell epitope of an antigen and an oxidoreductase motif.

### BACKGROUND OF THE INVENTION

Several strategies have been described to prevent the generation of an unwanted immune response against an antigen. WO2008/017517 describes a strategy using peptides comprising an MHC class II T cell epitope of a given antigenic protein and an oxidoreductase motif. These peptides convert CD4+ T cells into a cell type with cytolytic properties called cytolytic CD4+ T cells. These cells are capable to kill via triggering apoptosis those antigen presenting cells (APC), which present the antigen from which the peptide is derived. WO2008/017517 demonstrates this concept for allergies and auto-immune diseases such as type 1 diabetes. Herein insulin can act as an auto-antigen.
WO2009101207 and Carlier et al. (2012) Plos one 7,10 e45366 further describe the antigen specific cytolytic cells in more detail.
WO2016059236 discloses further modified peptides wherein an additional histidine is present in the proximity of the oxidoreductase motif.
WO2018162498 further discloses a peptide comprising an oxidoreductase motif with an additional histidine and a MHCII T cell epitope from insulin and its use in the treatment of type 1 diabetes (T1D).

However, even when taking into account the above, little is known regarding the actual effect of such peptides in patients during treatment and there remains a need for improving the therapeutic effect of such immunogenic peptides.

### SUMMARY OF THE INVENTION

The present invention provides improved methods for treating auto-immune diseases with immunogenic peptides comprising an epitope of an auto-antigen and an oxidoreductase motif.

The inventors have found that in patients, the level of responsiveness can be increased by adjusting the treatment and dosage scheme of administering said immunogenic peptides.

The invention hence provides the following aspects:
1. An immunogenic peptide with a length of between 9 and 50 amino acids for use in preventing or treating a disease or disorder selected from: an auto-immune disorder, a demyelinating disorder, allograft or transplant rejection, a tumor or cancer, an infection with an intracellular pathogen, an immune response to a soluble allofactor, an immune response to an allergen exposure, or an immune response to a viral vector used for gene therapy or gene vaccination in a subject, said peptide comprising an oxidoreductase motif and, separated from this motif by 0 to 7 amino acids, a T cell epitope sequence of an antigen involved in said disease or disorder, wherein said oxidoreductase motif comprises the motif: Zₘ-[CST]-Xₙ-C- (SEQ ID NO: 12 to 36) or Zₘ-C-Xₙ-[CST]- (SEQ ID NO: 37 to 61),

wherein n is an integer from 0 to 6, preferably 2, 1, 0, or 3.

wherein m is for an integer from 0 to 2, in which C stands for cysteine, S for serine, T for threonine, X for any amino acid and Z for any amino acid, preferably a basic amino acid, wherein said immunogenic peptide is administered in at least 5 doses of from 300 to 1500 μg of said immunogenic peptide with an interval of from about 12 days to about 28 days between two doses.

In a preferred embodiment said administration is done through intramuscular or subcutaneous injection.

In said general formula of the oxidoreductase motif, the hyphen (-) indicates the point of attachment of the oxidoreductase motif to the N-terminal end of the linker or the T-cell epitope, or to the C-terminal end of the linker or the T cell epitope.

In a preferred embodiment, the T cell epitope in said immunogenic peptide is not, or does not comprise, an amino acid sequence selected from the group consisting of: MHC class II T cell epitopes FLRVPCWKI (SEQ ID NO: 4), and FLRVPSWKI (SEQ ID NO: 5), or NKT cell epitopes FLRVPCW (SEQ ID NO: 10), and FLRVPSW (SEQ ID NO: 11).

In a preferred embodiment, said oxidoreductase motif is not part of a repeat of the standard C-XX-[CST] (SEQ ID NO: 1) or [CST]-XX-C(SEQ ID NO 2) oxidoreductase motifs such as repeats of said motif which can be spaced from each other by one or more amino acids (e.g. CXXC X CXXC X CXXC (SEQ ID NO: 6)), as repeats which are adjacent to each other (CXXCCXXCCXXC (SEQ ID NO: 7)) or as repeats which overlap with each other CXXCXXCXXC (SEQ ID NO: 8) or CXCCXCCXCC (SEQ ID NO: 9)), especially when n is 0 or 1 and m is 0 in the general formula as defined in aspect 1.

In a preferred embodiment, the antigen is an autoantigen.
2. The immunogenic peptide for use according to aspect 1, wherein said immunogenic peptide is administered through intramuscular or subcutaneous injection of 6 doses of from 300 to 1500 μg of said immunogenic peptide with an interval of about 12 to 28 days between two doses.
3. The immunogenic peptide for use according to aspect 1 or 2, wherein each of said doses of from 300 to 1500 μg of said immunogenic peptide is administered with an interval of about 12 to about 16 days, or about 2 weeks between two doses.
4. The immunogenic peptide for use according to any one of aspects 1 to 3, wherein each dose contains:
   from 300 to 600 μg of said immunogenic peptide;
   from 600 to 800 μg of said immunogenic peptide;
   from 800 to 1000 μg of said immunogenic peptide;
   from 1000 to 1200 μg of said immunogenic peptide; or
   from 1200 to 1500 μg of said immunogenic peptide.

In a preferred embodiment of said aspect, said dose contains 450 or 1350 μg of said immunogenic peptide.

In an even more preferred embodiment, said dose is administered 6 times, with an interval of about 12 to about 16 days, or about 2 weeks between doses.
5. The immunogenic peptide for use according to any one of aspects 1 to 4, wherein a boost administration is performed of a dose of from 300 to 1500 μg of said immunogenic peptide at about week 22 to 30, counted from the start of the treatment.
6. The immunogenic peptide for use according to aspect 5, wherein said boost administration is performed at about week 22 to 26 counted from the start of the treatment.
7. The immunogenic peptide for use according to aspect 5 or 6, wherein said boost contains:
   from 300 to 600 μg of said immunogenic peptide;
   from 600 to 800 μg of said immunogenic peptide;
   from 800 to 1000 μg of said immunogenic peptide;
   from 1000 to 1200 μg of said immunogenic peptide; or
   from 1200 to 1500 μg of said immunogenic peptide.

In a preferred embodiment of said aspect, said dose contains 450 or 1350 μg of said immunogenic peptide, preferably at about week 23 to 25 of the treatment, more preferably around week 24 of the treatment.
8. The immunogenic peptide for use according to any one of aspects 1 to 7, wherein half of the dose is to be administered concomitantly in two sites (both upper arms, preferably in the region of the lateral part of the arms, more preferably midway between the elbow and the shoulder).
9. An in vitro method for analysing the response of a patient to the treatment of a disease or disorder selected from: an auto-immune disorder, a demyelinating disorder, allograft or transplant rejection, a tumor or cancer, an infection with an intracellular pathogen, an immune response to a soluble allofactor, an immune response to an allergen exposure, or an immune response to a viral vector used for gene therapy or gene vaccination in a subject, with an immunogenic peptide with a length of between 9 and 50 amino acids, said peptide comprising an oxidoreductase motif and, separated from this motif by 0 to 7 amino acids, an MHC class II T cell epitope sequence of an (auto)-antigen of involved in said disease or disorder, wherein said oxidoreductase motif comprises the motif: Zₘ-[CST]-Xₙ-C- (SEQ ID NO: 12 to 36) or Zₘ-C-Xₙ-[CST]- (SEQ ID NO: 37 to 61),

wherein n is an integer from 0 to 6, preferably 2, 1, 0, or 3.

wherein m is for an integer from 0 to 2, in which C stands for cysteine, S for serine, T for threonine, X for any amino acid and Z for any amino acid, preferably a basic amino acid,

wherein said method comprises the analysis of samples taken from a patient being treated with said immunogenic peptide at the following time points:
Day 0 of the treatment,
In week 11 to 13, such as in week 12 of the treatment,
In week 23 to 25, such as in week 24 of the treatment, and
In week 47 to 49, such as in week 48 of the treatment.
10. The method according to aspect 9, wherein additionally a sample of said patient is analysed about 8 to 10 weeks prior to the start of the treatment, preferably about 9 weeks prior to the start of said treatment.
11. The immunogenic peptide for use according to any one of aspects 1 to 8 or the method according to aspect 9 or 10, wherein said auto-immune disease is selected from the group consisting of: type-1-diabetes (T1D), multiple sclerosis (MS) neuromyelitis optica (NMO), rheumatoid arthritis (RA), psoriasis, polyarthritis, asthma, atopic dermatitis, scleroderma, ulcerative colitis, juveline diabetes, thyreoiditis, Grave's disease, Systemic Lupus Erythromatosis (SLE), Sjogren syndrome, anemia perniciosa, chronic active hepatitis, transplant rejection and cancer.
12. The immunogenic peptide for use according to any one of aspects 1 to 8 or the method according to any one of aspects 9 to 11, wherein the said (auto)antigen does not naturally comprise an oxidoreductase motif within 11 amino acids N- or C-terminally adjacent to said epitope.
13. The immunogenic peptide for use according to any one of aspects 1 to 8 or the method according to any one of aspects 9 to 12, wherein in said immunogenic peptide said epitope does not naturally comprise an oxidoreductase motif in its sequence.
14. The immunogenic peptide for use according to any one of aspects 1 to 8 or the method according to any one of aspects 9 to 13, wherein in said immunogenic peptide the T-cell epitope is an MHC class I or II T-cell epitope or an NKT cell epitope.
An MHC class II epitope typically has a length of between 7 and 20 amino acids in length, more usually between 8 and 20 or 9 and 20 amino acids in length, even more preferably between 7 and 17, between 8 and 17, between 9 and 17, between 10 and 17, between 11 and 17, between 12 and 17, between 13 and 17 amino acids, such as between 14 and 16 amino acids. Peptides which bind to MHC class II molecules can also be longer since these peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.
An NKT cell epitope can be recognized and bound by a receptor at the cell surface of an NKT cell, in particular by CD1d molecules. Such an epitope typically has a length of between 7 and 20 amino acids, more usually between 7 and 17 amino acids in length, even more preferably between 8 and 17, between 9 and 17, between 10 and 17, between 11 and 17, between 12 and 17, between 13 and 17 amino acids, such as between 14 and 16 amino acids. Such epitopes typically have a motif [FWHY]-XX-[ILMV]-XX-[FWTHY] [SEQ ID NO: 62] or [FW]-XX-[ILMV]-XX-[FW] [SEQ ID NO: 63].
15. The immunogenic peptide for use according to any one of aspects 1 to 8 or the method according to any one of aspects 9 to 14, wherein in said immunogenic peptide the oxidoreductase motif is located N-terminally from the linker or the epitope, or C-terminally from the linker or the epitope, preferably N-terminally from the linker or the epitope, and/or wherein the oxidoreductase motif is located at the N-terminal or C-terminal end of the immunogenic peptide.
16. The immunogenic peptide for use according to any one of aspects 1 to 8 or the method according to any one of aspects 9 to 15, wherein in said immunogenic peptide said T cell epitope of an antigenic protein is an NKT cell epitope or an MHC class II T cell epitope, preferably wherein when said T cell epitope of an antigenic protein is an NKT cell epitope, it has a length of between 7 and 25 amino acids; or wherein when said T cell epitope of an antigenic protein is an MHC class II T cell epitope, it has a length of between 9 and 25 amino acids.
17. The immunogenic peptide for use according to any one of aspects 1 to 8 or the method according to any one of aspects 9 to 16, wherein said immunogenic peptide having an NKT epitope has a length of between 7 and 50 amino acids, and/or wherein said immunogenic peptide comprising an MHC class II T cell epitope has a length of between 9 and 50 amino acids.
18. The immunogenic peptide for use according to any one of aspects 1 to 8 or the method according to any one of aspects 9 to 17, wherein in said immunogenic peptide the linker between the oxidoreductase motif and the T cell epitope is of between 0 and 4 amino acids.
19. The immunogenic peptide for use according to any one of aspects 1 to 8 or the method according to any one of aspects 9 to 18, wherein in said immunogenic peptide said oxidoreductase motif with the sequence: Zₘ-[CST]-Xₙ-C- (SEQ ID NO: 12 to 36) or Zₘ-C-Xₙ-[CST]- (SEQ ID NO 37 to 61) as defined in aspect 1, is selected from the following amino acid motifs: (a) Zₘ-[CST]-Xₙ-C- or Zₘ-C-Xₙ-[CST]-,

wherein n is 0, and wherein m is an integer selected from 0, 1, or 2,

wherein Z is any amino acid, preferably a basic amino acid preferably selected from: H, K, R, and a non-natural basic amino acid as defined herein, such as L-ornithine, more preferably K or H, most preferably K;
(b) Zₘ-[CST]-Xₙ-C- or Zₘ-C-Xₙ-[CST]-,

wherein n is 1, wherein X is any amino acid, preferably a basic amino acid selected from: H, K, R, and a non-natural basic amino acid such as L-ornithine, more preferably K or R,

wherein m is an integer selected from 0, 1, or 2, wherein Z is any amino acid, preferably a basic amino acid preferably selected from: H, K, R, and a non-natural basic amino acid as defined herein, such as L-ornithine, more preferably K or H, most preferably K;
(c) Zₘ-[CST]-Xₙ-C- or Zₘ-C-Xₙ-[CST]-, wherein n is 2, thereby creating an internal X¹X² amino acid couple within the oxidoreductase motif, wherein X is any amino acid, preferably wherein at least one X is a basic amino acid selected from: H, K, R, and a non-natural basic amino acid such as L-ornithine, more preferably K or R, wherein m is an integer selected from 0, 1, or 2, wherein Z is any amino acid, preferably a basic amino acid preferably selected from: H, K, R, and a non-natural basic amino acid as defined herein, such as L-ornithine, more preferably K or H, most preferably K;
(d) Zₘ-[CST]-Xₙ-C- or Zₘ-C-Xₙ-[CST]-, wherein n is 3, thereby creating an internal X¹X2X³ amino acid stretch within the oxidoreductase motif, wherein X is any amino acid, preferably wherein at least one X is a basic amino acid selected from: H, K, R, and a non-natural basic amino acid such as L-ornithine, more preferably K or R, wherein m is an integer selected from 0, 1, or 2, wherein Z is any amino acid, preferably a basic amino acid preferably selected from: H, K, R, and a non-natural basic amino acid as defined herein, such as L-ornithine, more preferably K or H;
(e) Zₘ-[CST]-Xₙ-C- or Zₘ-C-Xₙ-[CST]-, wherein n is 4, thereby creating an internal X¹X2X³X⁴(SEQ ID NO: 64) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K; (f) Zₘ-[CST]-Xₙ-C- or Zₘ-C-Xₙ-[CST]-, wherein n is 5, thereby creating an internal X¹X2X³X4X⁵ (SEQ ID NO: 65) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K; (g) Zₘ-[CST]-Xₙ-C- or Zₘ-C-Xₙ-[CST]-, wherein n is 6, thereby creating an internal X¹X2X³X4X⁵X⁶(SEQ ID NO: 66) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K; or (h) Zₘ-[CST]-Xₙ-C- or Zₘ-C-Xₙ-[CST]-, wherein n is 0 to 6 and wherein m is 0, and wherein one of the C or [CST] residues has been modified so as to carry an acetyl, methyl, ethyl or propionyl group, either on the N-terminal amide of the amino acid residue of the motif or on the C-terminal carboxy group.

In preferred embodiments of such a motif, n is 2, and m is 1 or 2, wherein the internal X¹X2, each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹or X² in said motif is a basic amino acid selected from: H, K, or R, or a non-natural basic amino acid as defined herein, such as L-ornithine. In another example of the motif, at least one of X¹or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Preferably said modification results in an N-terminal acetylation of the first cysteine in the motif (N-acetyl-cysteine).
20. The immunogenic peptide for use according to any one of aspects 1 to 8 or the method according to any one of aspects 9 to 19, wherein said immunogenic peptide has an oxidoreductase motif which comprises the sequence CC, KCC, RCC, CRC, CKC, KCRC (SEQ ID NO: 154), KCKC (SEQ ID NO: 152), KCHC (SEQ ID NO: 225), RCRC (SEQ ID NO: 156), RCKC (SEQ ID NO: 226), CPYC (SEQ ID NO: 227), HCPYC (SEQ ID NO: 228), KCPYC (SEQ ID NO: 229), RCPYC (SEQ ID NO: 230), CRPYC (SEQ ID NO: 231), CPRYC (SEQ ID NO: 232), CPYRC (SEQ ID NO: 233), CKPYC (SEQ ID NO: 234), CPKYC (SEQ ID NO: 235), CPYKC (SEQ ID NO: 236), RCRPYC (SEQ ID NO: 237), RCPRYC (SEQ ID NO: 238), RCPYRC (SEQ ID NO: 239), RCKPYC (SEQ ID NO: 240), RCPKYC (SEQ ID NO: 241), RCPYKC (SEQ ID NO: 242), KCRPYC (SEQ ID NO: 243), KCPRYC (SEQ ID NO: 244), KCPYRC (SEQ ID NO: 245), KCKPYC (SEQ ID NO: 246), KCPKYC (SEQ ID NO: 247), or KCPYKC (SEQ ID NO: 248).
21. The immunogenic peptide for use according to any one of aspects 1 to 8 or the method according to any one of aspects 9 to 20, wherein the auto-immune disease is T1D and wherein the T-cell epitope in said peptide is an MHC class II T cell or NKT cell epitope from (pro-)insulin or C-peptide, preferably wherein the amino acid sequence of said epitope is defined by the amino acid sequence LALEGSLQK [SEQ ID NO: 3].
22. The immunogenic peptide for use according to any one of aspects 1 to 8 or the method according to any one of aspects 9 to 21, wherein said patients are homozygous or heterozygous HLA type DR3 or DR4 positive.
23. The immunogenic peptide for use according to any one of aspects 1 to 8 or the method according to any one of aspects 9 to 22, wherein said peptide comprises a sequence selected from the group consisting of: Cxx[CST]SLQPLALEGSLQK [SEQ ID NO: 67], [CST]xxCSLQPLALEGSLQK [SEQ ID NO:68], CxxCSLQPLALEGSLQK [SEQ ID NO: 69], HCxx[CST]SLQPLALEGSLQK [SEQ ID NO:70], H[CST]xxCSLQPLALEGSLQK [SEQ ID NO:71], HCxxCSLQPLALEGSLQK [SEQ ID NO: 72], and HCPYCSLQPLALEGSLQKRG [SEQ ID NO: 73].
24. The immunogenic peptide for use according to any one of aspects 1 to 8 or the method according to any one of aspects 9 to 20, wherein said antigen is an auto-antigen, an allergen, a soluble allofactor, an alloantigen shed by the graft, an antigen of an intracellular pathogen, an antigen of a viral vector used for gene therapy or gene vaccination, a tumor-associated antigen or an allergen.

### Exemplary Antigens can be

myelin antigens, neuronal antigens, and astrocyte-derived antigens, for example: Myelin Oligodendrocyte Glycoprotein (MOG), Myelin basic protein (MBP), Proteolipid protein (PLP), Oligodendrocyte-specific protein (OSP), myelin-associated antigen (MAG), myelin-associated oligodendrocyte basic protein (MOBP), and 2′,3′-cyclic-nucleotide 3′-phosphodiesterase (CNPase), S100P protein or transaldolase H autoantigens in case of MS (Riedhammer and Weissert, 2015; Front Immunol. 2015; 6: 322), preferably MOG, MBP, PLP and MOBP.
ADAMTSL5, PLA2G4D, Keratin, such as Keratin 14 or Keratin 17, an antigen from Triticum aestivum, Pso p27, cathelicidin antimicrobial peptide, ceutrophil defensin 1 and LL37, preferably LL37 in the case of psoriasis (Jason E. Hawkes et al., 2017: Current Dermatology Reports volume 6, pages 104-112).
allergens such as those derived from pollen, spores, dust mites, and pet dander in case of asthma.
tumour or cancer associated antigens such as oncogenes, proto-oncogenes, viral proteins, surviving factors or clonotypic or idiotypic determinants in case of cancer. Specific examples are: MAGE (melanoma-associated gene) products were shown to be spontaneously expressed by tumour cells in the context of MHC class I determinants, and as such, recognised by CD8+ cytolytic T cells. However, MAGE-derived antigens, such as MAGE-3, are also expressed in MHC class II determinants and CD4+ specific T cells have been cloned from melanoma patients (Schutz et al. (2000) Cancer Research 60: 6272-6275; Schuler-Thurner et al. (2002) J. Exp. Med. 195: 1279-1288). Peptides presented by MHC class II determinants are known in the art. Other examples include the gp100 antigen expressed by the P815 mastocytoma and by melanoma cells (Lapointe (2001; J. Immunol. 167: 4758-4764; Cochlovius et al. (1999) Int. J. Cancer, 83: 547-554). Proto-oncogenes include a number of polypeptides and proteins which are preferentially expressed in tumours cells, and only minimally in healthy tissues. Cyclin D1 is cell cycle regulator which is involved in the G1 to S transition. High expression of cyclin D1 has been demonstrated in renal cell carcinoma, parathyroid carcinomas and multiple myeloma. A peptide encompassing residues 198 to 212 has been shown to carry a T cell epitope recognised in the context of MHC class II determinants (Dengiel et al. (2004) Eur. J. of Immunol. 34: 3644-3651). Survivin is one example of a factor inhibiting apoptosis, thereby conferring an expansion advantage to survivin-expressing cells. Survivin is aberrantly expressed in human cancers of epithelial and hematopoietic origins and not expressed in healthy adult tissues except the thymus, testis and placenta, and in growth-hormone stimulated hematopoietic progenitors and endothelial cells. Interestingly, survivin-specific CD8+ T cells are detectable in blood of melanoma patients. Survivin is expressed by a broad variety of malignant cell lines, including renal carcinoma, breast cancer, and multiple myeloma, but also in acute myeloid leukemia, and in acute and chronic lymphoid leukemia (Schmidt (2003) Blood 102: 571-576). Other examples on inhibitors of apoptosis are Bcl2 and spi6. Idiotypic determinants are presented by B cells in follicular lymphomas, multiple myeloma and some forms of leukemia, and by T cell lymphomas and some T cell leukemias. Idiotypic determinants are part of the antigen-specific receptor of either the B cell receptor (BCR) or the T cell receptor (TCR). Such determinants are essentially encoded by hypervariable regions of the receptor, corresponding to complementarity-determining regions (CDR) of either the VH or VL regions in B cells, or the CDR3 of the beta chain in T cells. As receptors are created by the random rearrangement of genes, they are unique to each individual. Peptides derived from idiotypic determinants are presented in MHC class II determinants (Baskar et al. (2004) J. Clin. Invest. 113: 1498-1510). Some tumours are associated with expression of virus-derived antigens. Thus, some forms of Hodgkin disease express antigens from the Epstein-Barr virus (EBV). Such antigens are expressed in both class I and class II determinants. CD8+ cytolytic T cells specific for EBV antigens can eliminate Hodgkin lymphoma cells (Bollard et a/. (2004) J. Exp. Med. 200: 1623-1633). Antigenic determinants such as LMP-1 and LMP-2 are presented by MHC class II determinants.
transplant-specific antigens in case of transplant rejection, which will obviously be dependent on the type of tissue or organ being transplanted. Examples can be tissues such as cornea, skin, bones (bone chips), vessels or fascia; organs such as kidney, heart, liver, lungs, pancreas or intestine; or even individual cells such as pancreatic islet cells, alpha cells, beta cells, muscle cells, cartilage cells, heart cells, brain cells, blood cells, bone marrow cells, kidney cells and liver cells. Specific exemplary antigens involved in transplantation rejection are minor histocompatibility antigens, major histocompatibility antigens or tissue-specific antigens. Where the alloantigenic protein is a major histocompatibility antigen, this is either an MHC class I-antigen or an MHC class Il-antigen. An important point to keep in mind is the variability of the mechanisms by which alloantigen-specific T cells recognize cognate peptides at the surface of APC. Alloreactive T cells can recognize either alloantigen-determinants of the MHC molecule itself, an alloantigen peptide bound to a MHC molecule of either autogenic or allogeneic source, or a combination of residues located within the alloantigen-derived peptide and the MHC molecule, the latter being of autogenic or allogeneic origin. Examples of minor histocompatibility antigens are those derived from proteins encoded by the HY chromosome (H-Y antigens), such as Dby. Other examples can be found in, for instance, Goulmy E, Current Opinion in Immunology, vol 8, 75-81, 1996 (see Table 3 therein in particular). It has to be noted that many minor histocompatibility antigens in man have been detected via their presentation into MHC class I determinants by use of cytolytic CD8+ T cells. However, such peptides are derived by the processing of proteins that also contain MHC class II restricted T cell epitopes, thereby providing the possibility of designing peptides of the present invention. Tissue-specific alloantigens can be identified using the same procedure. One example of this is the MHC class I restricted epitope derived from a protein expressed in kidneys but not in spleen and capable of eliciting CD8+ T cells with cytotoxic activity on kidney cells (Poindexter et al, Journal of Immunology, 154: 3880-3887, 1995).

More preferably, said antigenic protein is an autoantigen involved in type-1 diabetes (T1D), demyelinating disorders such as multiple sclerosis (MS) or neuromyelitis optica (NMO), or in rheumatoid arthritis (RA).

### Non-Limiting Examples of Autoantigens Involved in TiD.

Source: Mallone R et al., Clin Dev Immunol. 2011:513210.

| | | Examples ofl |
|---|---|---|
| Auto- | UniProtKB | T-cel |
| antigen | identifier | epitopes |
| Insulin | P01308 | LALEGSLQK |
| | | (SEQ ID |
| | | NO: 3) |
| GAD65 | Q9UGI5 | |
| GAD67 | Q99259 | |
| IA-2 (ICA512) | Q16849 | |
| IA-2 | Q92932 | |
| (beta/phogrin) | | |
| IGRP | Q9NQR9 | |
| Chromogranin | P10645 | |
| ZnT8 | Q8IWU4 | |
| HSP-60 | P10809 | |

### Non-Limiting Examples of Autoantigens Involved in MS.

| | | | Examples of |
|---|---|---|---|
| | UniProtKB | | T-cell |
| Auto-antigen | identifier | Comments | epitopes |
| Myelin oligodendrocyte | Q16653 | Immunodominant | FLRVPCWKI |
| glycoprotein (MOG) | | epitopes in the following | (SEQ ID NO: 4) |
| | | regions: amino acids 1- | or FLRVPSWKI |
| | | 22, 35-55 and 92-106. | (SEQ ID NO: 5) |
| Myelin basic protein | P02686 | Immunodominant | |
| (MBP) | | epitopes in the amino | |
| | | acids 85-99 region. | |
| Proteolipid protein | P60201 | Immunodominant | |
| (PLP) | | epitopes in the following | |
| | | regions: amino acids | |
| | | 31-70, 91-120 and | |
| | | 178-228. | |
| Myelin- | Q13875 | Immunodominant | |
| oligodendrocytic basic | | epitopes in the amino | |
| protein (MOBP) | | acids 15-36 region. | |
| Oligodendrocyte- | O75508 | Immunodominant | |
| specific protein (OSP) | | epitopes in the amino | |
| | | acids 179-207 region. | |

### Non-Limiting Examples of Autoantigens Involved in RA.

| Auto-antigen | UniProtKB identifier |
|---|---|
| GRP78 | P11021 |
| HSP60 | P10809 |
| 60 kDa chaperonin 2 | P9WPE7 |
| Gelsolin | P06396 |
| Chitinase-3-like protein 1 | P36222 |
| Cathepsin S | P25774 |
| Serum albumin | P02768 |
| Cathepsin D | P07339 |

### Example of Autoantigens Involved in NMO

| | UniProtKB | | Examples |
|---|---|---|---|
| Auto- | identi- | | of T-cell |
| antigen | fier | Comments | epitopes |
| Myelin | Q16653 | Immunodominant | FLRVPCWKI |
| oligo- | | epitopes | (SEQ |
| dendrocyte | | in the | ID NO: 4) |
| glyco- | | following | or |
| protein | | regions: | FLRVPSWKI |
| (MOG) | | amino acids | (SEQ |
| | | 1-22, 35-55 | ID NO: 5) |
| | | and 92-106. | |

In some embodiments, in any one of the aspects defined herein, the epitope in said peptide is not a sequence derived from the MOG antigen amino acid sequence.

In some embodiments, in any one of the aspects defined herein, said disease or disorder is not MS.

In some embodiments, in any one of the aspects defined herein, said disease or disorder is not a disease that is known to be treated by fumarate, or is not a fumarate-related disease or disorder as defined herein elsewhere.
25. The immunogenic peptide for use according to any one of aspects 1 to 8 or the method according to any one of aspects 9 to 20, wherein the auto-immune disease is MS and wherein the T-cell epitope in said peptide is an MHC class II T cell or NKT cell epitope from an antigenic protein selected from the group comprising: Myelin Oligodendrocyte Glycoprotein (MOG), Myelin basic protein (MBP), Proteolipid protein (PLP), myelin-associated antigen (MAG), Oligodendrocyte-specific protein (OSP), myelin-associated oligodendrocyte basic protein (MOBP), 2′,3′-cyclic-nucleotide 3′-phosphodiesterase (CNPase), S100P protein and transaldolase H, preferably MOG.

In an alternative embodiment, in the immunogenic peptide for use according to any one of aspects 1 to 8 or the method according to any one of aspects 9 to 20, the auto-immune disease is MS and the T-cell epitope in said peptide is an MHC class II T cell or NKT cell epitope from an antigenic protein selected from the group comprising: Myelin basic protein (MBP), Proteolipid protein (PLP), myelin-associated antigen (MAG), Oligodendrocyte-specific protein (OSP), myelin-associated oligodendrocyte basic protein (MOBP), 2′,3′-cyclic-nucleotide 3′-phosphodiesterase (CNPase), S100B protein and transaldolase H.
26. The immunogenic peptide for use according to any one of aspects 1 to 8, or the method according to any one of aspects 9 to 20, wherein the auto-immune disease is NMO and wherein the T-cell epitope in said peptide is an MHC class II T cell or NKT cell epitope from Myelin Oligodendrocyte Glycoprotein (MOG).
27. The immunogenic peptide for use according to any one of aspects 1 to 8 or the method according to any one of aspects 9 to 20, wherein the auto-immune disease is RA and wherein the T-cell epitope in said peptide is an MHC class II T cell or NKT cell epitope from an antigenic protein selected from the group comprising: GRP78, HSP60, 60 kDa chaperonin 2, Gelsolin, Chitinase-3-like protein 1, Cathepsin S, Serum albumin, vinculin, and Cathepsin D.
28. The immunogenic peptide for use according to any one of aspects 1 to 8 or the method according to any one of aspects 9 to 20, wherein the auto-immune disease is Psoriasis and wherein said antigenic protein is selected from the group consisting of: ADAMTSL5, PLA2G4D, Keratin, such as Keratin 14 or Keratin 17, an antigen from Triticum aestivum, Pso p27, cathelicidin antimicrobial peptide, ceutrophil defensin 1 and LL37, preferably LL37.
29. The immunogenic peptide for use according to any one of aspects 1 to 8 or the method according to any one of aspects 9 to 28, wherein when said auto-immune disease is MS, NMO or psoriasis, said treatment does not include treatment with a fumarate compound as defined herein or a derivative thereof.

In a preferred embodiment, the T cell epitope of said peptide is not a T-cell epitope of an antigenic protein or antigen involved in a fumarate-related or fumarate-treated disease or disorder, more preferably an MHC class I or II molecule or an NKT cell epitope.
26. The immunogenic peptide for use according to any one of aspects 1 to 8 or the method according to any one of aspects 9 to 20 and 24 or 25, wherein said epitope is derived from the Myelin-oligodendrocyte glycoprotein (MOG) antigen amino acid sequence. More preferably said epitope is selected from the group comprising amino acid residues: 40-60, 41-55, 43-57, 44-58, 45-59, and 35-55 of the mature MOG amino acid sequence defined by SEQ ID NO: 74:

GQFRVIGPRHPIRALVGDEVELPCRISPGKNATGMEVGW
YRPPFSRVVHLYRNGKDQDGDQAPEYRGRTELLKDAIGE
GKVTLRIRNVRFSDEGGFTCFFRDHSYQEEAAMELKVED
PFYWVSPGVLVLLAVLPVLLLQITVGLIFLCLQYRLRGK
LRAEIENLHRTFDPHFLRVPCWKITLFVIVPVLGPLVAL
IICYNWLHRRLAGQFLEELRNPF,

Such as those selected from the group comprising:

(SEQ ID NO: 75)
YRPPFSRVVHLYRNGKDQDGD
(SEQ ID NO: 76)
RPPFSRVVHLYRNGK
(SEQ ID NO: 77)
PFSRVVHLYRNGKDQ
(SEQ ID NO: 78)
FSRVVHLYRNGKDOD
(SEQ ID NO: 79)
SRVVHLYRNGKDQDG
(SEQ ID NO: 80)
VVHLYRNGK
(mouse SEQ ID NO: 81)
MEVGWYRSPFSRVVHLYRNGK,
(human SEQ ID NO: 82)
MEVGWYRPPFSRVVHLYRNGK,
(mouse SEQ ID NO: 83)
YRSPFSRVV,
ands
(human SEQ ID NO: 84)
YRPPFSRVV,

or combinations thereof.

In a preferred embodiment of aspect 6 or 7, said immunogenic peptide comprises a T-cell epitope having or comprising the T-cell epitope defined by the following sequence: FLRVPCWKI (SEQ ID NO: 4) or FLRVPSWKI (SEQ ID NO:5), or said immunogenic peptide comprises or consists essentially of the amino sequence:

(SEQ ID NO: 85)
HCPYCVRYFLRVPSWKITLF,
(SEQ ID NO: 86)
HCPYCVRYFLRVPCWKITLF,
(SEQ ID NO: 87)
KHCPYCVRYFLRVPSWKITLFKK,
or
(SEQ ID NO: 88)
KHCPYCVRYFLRVPCWKITLFKK

27. The immunogenic peptide for use according to any one of aspects 1 to 8 or the method according to any one of aspects 9 to 20 and 24, wherein the epitope in said immunogenic or tolerogenic peptide is derived from the myelin proteolipid protein (also called proteolipid protein (PLP) or lipohilin) antigen amino acid sequence. More preferably, with reference to patent application WO2014111841, said epitope is selected from the group comprising amino acid residues: 36-61, 179-206, 207-234, 39-57, 180-198, 208-222, 39-53, 42-56, 43-57, 180-194, 181-195, 182-196, 183-197, 184-198, 208-222, 36-61, 179-206, and 207-234 of the PLP amino acid sequence defined by SEQ ID NO: 89 (UniProtKB - P60201 (MYPR_HUMAN)):

MGLLECCARCLVGAPFASLVATGLCFFGVALFCGCGHEALTGTEKL
IETYFSKNYQDYEYLINVIHAFQYVIYGTASFFFLYGALLLAEGF
YTTGAVRQIFGDYKTTICGKGLSATVTGGQKGRGSRGQHQAHSLE
RVCHCLGKWLGHPDKFVGITYALTVVWLLVFACSAVPVYIYFNTW
TTCQSIAFPSKTSASIGSLCADARMYGVLPWNAFPGKVCGSNLLS
ICKTAEFQMTFHLFIAAFVGAAATLVSLLTFMIAATYNFAVLKLM
GRGTKF.

Such as those selected from the group comprising:

PLP 36-61:
(SEQ ID NO: 90)
HEALTGTEKLIETYFSKNYQDYEYLI;
PLP 179-206:
(SEQ ID NO: 91)
TWTTCQSIAFPSKTSASIGSLCADARMY;
PLP 207-234:
(SEQ ID NO: 92)
GVLPWNAFPGKVCGSNLLSICKTAEFQM;
PLP 39-57:
(SEQ ID NO: 93)
LTGTEKLIETYFSKNYQDY
PLP 180-198:
(SEQ ID NO: 94)
WTTCQSIAFPSKTSASIGS
PLP 208-222:
(SEQ ID NO: 95)
VLPWNAFPGKVCGSN
PLP 39-53:
(SEQ ID NO: 96)
LTGTEKLIETYFSKN
PLP 42-56:
(SEQ ID NO: 97)
TEKLIETYFSKNYQD
PLP 43-57:
(SEQ ID NO: 98)
EKLIETYFSKNYQDY
PLP 180-194:
(SEQ ID NO: 99)
WTTCQSIAFPSKTSA
PLP 181-195:
(SEQ ID NO: 100)
TTCQSIAFPSKTSAS
PLP 182-196:
(SEQ ID NO: 101)
TCQSIAFPSKTSASI
PLP183-197:
(SEQ ID NO: 102)
CQSIAFPSKTSASIG
PLP 184-198:
(SEQ ID NO: 103)
QSIAFPSKTSASIGS
PLP 208-222:
(SEQ ID NO: 104)
VLPWNAFPGKVCGSN
PLP 36-61:
(SEQ ID NO: 105)
HEALTGTEKLIETYFSKNYQDYEYLI
PLP 179-206:
(SEQ ID NO: 106)
TWTTCQSIAFPSKTSASIGSLCADARMY
and
PLP 207-234:
(SEQ ID NO: 107)
GVLPWNAFPGKVCGSNLLSICKTAEFQM

or combinations thereof.
28. The immunogenic peptide for use according to any one of aspects 1 to 8 or the method according to any one of aspects 9 to 20 and 24, wherein the epitope in said immunogenic or tolerogenic peptide is derived from the myelin basic protein (MBP) antigen amino acid sequence. More preferably said MBP epitope is selected from the group comprising the following sequences:

(SEQ ID NO: 108)
PRHRDTGILDSIGRF
(SEQ ID NO: 109)
ENPVVHFFKNIVTPRTP
(SEQ ID NO: 110)
RASDYKSAHKGFKGV
(SEQ ID NO: 111)
GFKGVDAQGTLSKIF
(SEQ ID NO: 112)
LGGRDSRSGSPMARR
(SEQ ID NO: 113)
TQDENPVVHFFKNIVTPRTP
(SEQ ID NO: 114)
TQDENPVVHFFKNIV
(SEQ ID NO: 115)
QDENPVVHFFKNIVT
(SEQ ID NO: 116)
DENPVVHFFKNIVTP
(SEQ ID NO: 117)
ENPVVHFFKNIVTPR
(SEQ ID NO: 118)
NPVVHFFKNIVTPRT
(SEQ ID NO: 119)
PVVHFFKNIVTPRTP
(SEQ ID NO: 120)
ASDYKSAHKGFKGVDAQGTLSKIFKLGG
(SEQ ID NO: 121)
ASDYKSAHKGFKGVD
(SEQ ID NO: 122)
SDYKSAHKGFKGVDA
(SEQ ID NO: 123)
DYKSAHKGFKGVDAQ
(SEQ ID NO: 124)
YKSAHKGFKGVDAQG
(SEQ ID NO: 125)
KSAHKGFKGVDAQGT
(SEQ ID NO: 126)
SAHKGFKGVDAQGTL
(SEQ ID NO: 127)
AHKGFKGVDAQGTLS
(SEQ ID NO: 128)
HKGFKGVDAQGTLSK
(SEQ ID NO: 129)
KGFKGVDAQGTLSKI
(SEQ ID NO: 130)
GFKGVDAQGTLSKIF
(SEQ ID NO: 131)
FKGVDAQGTLSKIFK
(SEQ ID NO: 132)
KGVDAQGTLSKIFKL
(SEQ ID NO: 133)
GVDAQGTLSKIFKLG
(SEQ ID NO: 134)
VDAQGTLSKIFKLGG,
and
(SEQ ID NO: 135)
LSRFSWGAEGQRPG,

or combinations thereof,

or any one or more of the fragments defined by amino acid residues 30-44, 80-94, 83-99, 81-95, 82-96, 83-97, 84-98, 110-124, 130-144, 131-158, 131-145, 140-148, 142-152, 132-146, 134-148,135-149, 136-150,137-151, 138-152,139-153, 140-154 and 133-147 of the MBP amino acid sequence defined by SEQ ID NO: 136 (UniProtKB - P02686-5 (MBP_HUMAN)):

MASQKRPSQRHGSKYLATASTMDHARHGFLPRHRDTGILDSIG
RFFGGDRGAPKRGSGKDSHHPARTAHYGSLPQKSHGRTQDENP
VVHFFKNIVTPRTPPPSQGKGRGLSLSRFSWGAEGQRPGFGYG
GRASDYKSAHKGFKGVDAQGTLSKIFKLGGRDSRSGSPMARR.

29. In any one of the aspects disclosed herein relating to methods of treatment or medical uses of the immunogenic peptide, said peptides can also be administered as a nucleic acid encoding said respective peptide. A nucleic acid encoding the immunogenic or tolerogenic peptide according to any one of the aspects or examples disclosed herein, preferably selected from isolated desoxyribonucleic acid (DNA), plasmid DNA (pDNA), coding DNA (cDNA), ribonucleic acid (RNA), messenger RNA (mRNA) or modified versions thereof, such as non-immunogenic mRNA comprising N(1)-methyl-pseudouridine (m1ψ). In some embodiments, said nucleic acid can be part of an expression cassette, optionally incorporated in a (viral) vector or plasmid that can be used for gene-therapy or can be present in the form of encapsulated or naked DNA or RNA to be administered according to techniques known in the pharmaceutical and gene therapeutic field.
30. In any one of the aspects disclosed herein relating to methods of treatment or medical uses of the immunogenic peptide, said peptide can be recognized by specific HLA types:
when said disease is T1D, said antigen is preferably recognized in the context of HLA-DRB1*03 and DRB1*04 haplotype groups. In the DRB1*03 group, two alleles are common, namely DRB1*0301 and DRB1*0302, but other alleles have been reported, such as DRB1*0303, DRB1*0304 and DRB1*0307. In the DRB1*04 group, ten major alleles can be found, namely: DRB1*0401, DRB1*0402, DRB1*0403, DRB1*0404, DRB1*0405, DRB1*0406, DRB1*0407, DRB1*0408, DRB1*0410 and DRB1*0411.
when said disease or disorder is MS, said antigen is preferably recognized in the context of HLA-DRB1*15:01, HLA-DRB1*03:01, HLA-DRB1*04:01, HLA-DRB1*07:01, HLA DRB5*0101, or DQ6 type of HLA. More preferred are patients having a HLA-DRB1* type 15:01;
when said disease or disorder is NMO, said antigen is preferably recognized in the context of HLA-DRB1*03:01 or HLA-DPB1*05:01 (for Asia); or
when said disease or disorder is RA, said antigen is preferably recognized in the context of HLA-DRB1*01:01, 04:01 or 04:04. 31. In a preferred embodiment, the T1D patients treated with the immunogenic peptide are DR4 positive (i.e. positive for one of the DRB1*04 haplotypes), more preferably the T1D patients treated with the immunogenic peptide are DR4 positive (i.e. positive for one of the DRB1*04 haplotypes) and DR3 negative (i.e. negative for all DRB1*03 haplotypes). Alternatively, the T1D patients treated with the immunogenic peptide are DR3 positive (i.e. positive for one of the DRB1*03 haplotypes). The term “HLA DR4 positive” encompasses both patients being heterozygous or homozygous HLA-DR4 positive. In one embodiment, said patients are also HLA-DR3 negative (HLA-DR3-). The term “HLA-DR3 negative” refers to patients being homozygous HLA-DR3 negative.
32. In any one of the aspects defined herein, said peptide can be administered as a pharmaceutical composition comprising said peptide and a pharmaceutically acceptable carrier and/or an adjuvant.
33. The invention also provides for methods of preventing or treating an auto-immune disorder, a demyelinating disorder, transplant rejection or cancer, comprising administering an immunogenic peptide as defined in any one of the aspects above, using the administration scheme of any one of the aspects above.
34. The invention also provides for the use of an immunogenic peptide as defined in any one of the aspects above for the manufacture of a medicament for preventing or treating an auto-immune disorder, a demyelinating disorder, transplant rejection or cancer, wherein said immunogenic peptide is administered according to the administration scheme of any one of the aspects above.
35. A method of preventing or treating an auto-immune disorder, a demyelinating disorder, transplant rejection or cancer, comprising the steps of administering to a subject in need thereof the immunogenic peptide as defined in any one of the preceding aspects or nucleic acid encoding such, wherein said immunogenic peptide or nucleic acid is administered according to the administration scheme of any one of the preceding aspects.

### BRIEF DESCRIPTION OF THE FIGURES

FIG. 1: schematic representation of the main clinical study and the sub clinical study performed with the investigational medicinal product (IMP). See text for details.

FIG. 2: represent the patient's journey from 9 weeks post diagnosis to week 48. DBS: Dry Blood Spot analysis. FUp: follow up. See text for details.

FIG. 3: represents net % of CD4+ T cells responding to proinsulin epitope C20-A1 stimulations in IMP (continuous line) and Placebo (dotted line) treated patient groups at various time points. The straight lines connect the mean values while the error bars show the standard errors at each time point. The p-values at individual time points compare the IMP and Placebo treated patient groups using Mann-Whitney-Wilcoxon U test. The p-values displayed on the right hand side of the line plots signify the dependence of mean response on time using repeated measures ANOVA.

### DETAILED DESCRIPTION OF THE INVENTION

The present invention will be described with respect to particular embodiments but the invention is not limited thereto but only by the claims. Any reference signs in the claims shall not be construed as limiting the scope. The following terms or definitions are provided solely to aid in the understanding of the invention. Unless specifically defined herein, all terms used herein have the same meaning as they would to one skilled in the art of the present invention. The definitions provided herein should not be construed to have a scope less than the one understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise. The term “any” when used in relation to aspects, claims or embodiments as used herein refers to any single one (i.e. anyone) as well as to all combinations of said aspects, claims or embodiments referred to.

The terms ‘comprising’, ‘comprises' and ‘comprised of’ as used herein are synonymous with ‘including’, ‘includes' or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints. The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed. More particularly, when referring to a period of time in weeks, the term ‘about’ refers to +/−2 days.

As used herein, the term “for use” as used in “composition for use in treatment of a disease” shall disclose also the corresponding method of treatment and the corresponding use of a preparation for the manufacture of a medicament for the treatment of a disease”.

The term “fumarate compound” as used herein refers to a compound of the general formula (I)

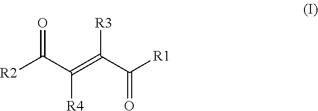

wherein R¹ and R² each independently are selected from the groups consisting of: OH, O⁻, and optionally substituted (C₁₋₁₀)alkoxy, preferably optionally substituted (C₁₋₆)alkoxy, or optionally substituted (C₁₋₃)alkoxy,

wherein R³ and R⁴ each independently are selected from the groups consisting of: H or deuterium,

wherein each group independently can be optionally substituted.

The term “peptide” as used herein refers to a molecule comprising an amino acid sequence of between 12 and 200 amino acids, connected by peptide bonds, but which can comprise non-amino acid structures.

Peptides according to the invention can contain any of the conventional 20 amino acids or modified versions thereof, or can contain non-naturally occurring amino-acids incorporated by chemical peptide synthesis or by chemical or enzymatic modification.

The term “antigen” as used herein refers to a structure of a macromolecule, typically protein (with or without polysaccharides) or made of proteic composition comprising one or more hapten (s) and comprising T cell epitopes.

The term “antigenic protein” as used herein refers to a protein comprising one or more T cell epitopes. An auto-antigen or auto-antigenic protein as used herein refers to a human or animal protein present in the body, which elicits an immune response within the same human or animal body.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B or T cell lymphocyte, and which is able, by said binding, to induce an immune response.

The term “T cell epitope” in the context of the present invention refers to an MHC class II T-cell epitope a dominant, sub-dominant or minor T cell epitope, i.e. a part of an antigenic protein that is specifically recognised and bound, when complexed with a MHC class II molecule, by a receptor expressed at the cell surface of a T lymphocyte, or refers to an NKT cell epitope. Whether an epitope is dominant, sub-dominant or minor depends on the immune reaction elicited against the epitope. Dominance depends on the frequency at which such epitopes are recognised by T cells and able to activate them, among all the possible T cell epitopes of a protein.

The T cell epitope is an epitope that is recognised and associates to MHC class II molecules, which consists of a sequence of +/−9 amino acids which fit in the groove of the MHC II molecule. Within a peptide sequence representing a T cell epitope, the amino acids in the epitope are numbered P1 to P9, amino acids N-terminal of the epitope are numbered P-1, P-2 and so on, amino acids C terminal of the epitope are numbered P+1, P+2 and so on. Peptides recognised by MHC class II molecules and not by MHC class I molecules are referred to as MHC class II restricted T cell epitopes. The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes: HLA-A, HLA-B, HLA-C, HLA-DPA1, HLA-DPB1, HLA-DQA1, HLAs DQB1, HLA-DRA, and HLA-DRB1. In humans, the MHC is divided into three regions: Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2). Class I MHC molecules are expressed on virtually all nucleated cells.

At the genetic level, the MHC class II cluster is located on the short arm of chromosome 6 (6p21). The cluster includes three classical class II genes (HLA-DP, -DQ and DR) and two non-classical class II genes (HLA-DM and -DO). The structure of MHC class II is achieved by the association of two membrane bound chains, called α and β, that create the antigen-binding cleft of MHC class II. Both α and β chains are encoded by distinct loci closely linked as pairs of α and β genes, i.e. DRα/DRβ, DQα/DQβ and DPα/DPβ. HLA-DP, -DQ and DR loci are highly polymorphic, especially in the antigen-binding pocket of the class II molecule. HLA-DP and -DQ contain polymorphisms in both the −α and −β chain genes (DPA, DPB, DQA and DQB). In HLA-DR, polymorphism concerns only the DR β chain (DRB gene). There are 9 DRB loci (numbered from DRB1 to DRB9), but only the DRB1 locus is found on all haplotypes, and hence constitutes the major determinant of classical DR serology (McCluskey et al, Current Protocols in Immunology (2017), 118, A.1S.1-A.1S.6).

Taking as an example the HLA-DRB1 group, literature has reported the existence of over 40 different haplotypes (Marsh et al, Tissue Antigens (2010), 75, p291). Of most relevance throughout the human population are the DRB1*03 and DRB1*04 haplotype groups. In the DRB1*03 group, two alleles are common, namely DRB1*0301 and DRB1*0302, but other alleles have been reported, such as DRB1*0303, DRB1*0304 and DRB1*0307. In the DRB1*04 group, ten major alleles can be found, namely: DRB1*0401, DRB1*0402, DRB1*0403, DRB1*0404, DRB1*0405, DRB1*0406, DRB1*0407, DRB1*0408, DRB1*0410 and DRB1*0411.

The term “DR4 positive” or “DR4+” used throughout the application indicates that the subject is positive for one of the DRB1*04 haplotypes. Similarly, the term “DR3 positive” or “DR3+” used throughout the application indicates that the subject is positive for one of the DRB1*03 haplotypes. The term “DR4 negative” or “DR4-” used throughout the application indicates that the subject does not have any of the DRB1*04 haplotypes. Similarly, the term “DR3 negative” or “DR3-” used throughout the application indicates that the subject does not have any of the DRB1*03 haplotypes.

HLA typing can be performed using techniques known in the art including, without limitation, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry, and sequencing by hybridization. Non-limiting examples of electrophoretic analysis include lab gel electrophoresis such as agarose or polyacrylamide gel electrophoresis, capillary electrophoresis, and denaturing gradient gel electrophoresis. Other methods for genotyping an individual at a polymorphic site in a marker include, e.g., the INVADER® assay from Third Wave Technologies, Inc., restriction fragment length polymorphism (RFLP) analysis, allele-specific oligonucleotide hybridization, a heteroduplex mobility assay, and single strand conformational polymorphism (SSCP) analysis. Alternatively, HLA typing can be performed by antibody testing.

Peptide fragments presented in the context of class I MHC molecules are recognised by CD8+ T lymphocytes (cytolytic T lymphocytes or CTLs). CD8+ T lymphocytes frequently mature into cytolytic effectors which can lyse cells bearing the stimulating antigen. Class II MHC molecules are expressed primarily on activated lymphocytes and antigen-presenting cells. CD4+ T lymphocytes (helper T lymphocytes or Th) are activated with recognition of a unique peptide fragment presented by a class II MHC molecule, usually found on an antigen-presenting cell like a macrophage or dendritic cell. CD4+ T lymphocytes proliferate and secrete cytokines such as IL-2, IFN-gamma and IL-4 that support antibody-mediated and cell mediated responses.

Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N-and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P-1, P-2 and so on, amino acids C-terminal of the epitope are numbered P+1, P+2 and so on.

The term “NKT cell epitope” refers to a part of an antigenic protein that is specifically recognized and bound by a receptor at the cell surface of an NKT cell. In particular, a NKT cell peptide epitope is an epitope bound by CD1d molecules, with motif [FWHY]-XX-[ILMV]-XX-[FWTHY] (SEQ ID NO: 62) or a more restrictive form thereof, such as [FW]-XX-[ILMV]-XX-[FW] (SEQ ID NO: 63). In this motif, F stands for phenylalanine, W for tryptophan, H for histidine, Y for tyrosine, I for isoleucine, L for leucine, M for methionine, V for valine, and X for any amino acid. [FWHY]indicates that either F, W, H or Y can occupy the first anchoring residue (P1), that the P4 position can be occupied by either I, L, M or V, and that P7 can be occupied by F, W, H or Y. It should be clear for the one skilled in the art that various combinations of these amino acid residues are possible.

The term “NKT cells” refers to cells of the innate immune system characterized by the fact that they carry receptors such as NK1.1 and NKG2D, and recognize peptide epitopes presented by the CD1d molecule. In the context of the present invention, NKT cells can belong to either the type 1 (invariant) or the type 2 subset, or to any of the less characterized NKT cells with more polymorphic T cell receptors than type 1 or type 2 NKT cells.

The “CD1d molecule” refers to a non-MHC derived molecule, expressed at the surface of various APCs, made of 3 alpha chains and an anti-parallel set of beta chains arranged into a deep hydrophobic groove opened on both sides and capable of presenting lipids, glycolipids or hydrophobic peptides to NKT cells.

The present invention provides methods for generating antigen-specific cytolytic CD4+ T cells either in vivo or in vitro and, independently thereof, methods to discriminate cytolytic CD4+ T cells from other cell populations such as Foxp3+ Tregs based on characteristic expression data.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the redox motif and the MHC class II epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a (poly)nucleotide sequence, which, when expressed in an appropriate environment, results in the generation of the relevant peptide sequence or a derivative or homologue thereof. Such polynucleotides or nucleic acids include the normal desoxyribonucleotide (DNA) or ribonucleotide (RNA) sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment. Such a nucleic acid encoding said immunogenic or tolerogenic peptide, is preferably selected from isolated desoxyribonucleic acid (DNA), plasmid DNA (pDNA), coding DNA (cDNA), ribonucleic acid (RNA), messenger RNA (mRNA) or modified versions thereof.

For therapeutic means, polynucleotides encoding the immunogenic peptides disclosed herein can be part of an expression system, cassette, plasmid or vector system such as viral and non-viral expression systems. Viral vectors known for therapeutic purposes are adenoviruses, adeno-associated viruses (AAVs), lentiviruses, and retroviruses. Non-viral vectors can be used as well and non-limiting examples include: transposon-based vector systems such as those derived from Sleeping Beauty (SB) or PiggyBac (PB). Nucleic acids can also be delivered through other carriers such as but not limited to nanoparticles, cationic lipids, liposomes etc.

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases. An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “type 1 diabetes” (T1D) or “diabetes type 1” (also known as “type 1 diabetes mellitus” or “immune mediated diabetes” or formerly known as “juvenile onset diabetes” or “insulin dependent diabetes”) is an autoimmune disorder that typically develops in susceptible individuals during childhood. At the basis of T1D pathogenesis is the destruction of most insulin-producing pancreatic beta-cells by an autoimmune mechanism. In short, the organism loses the immune tolerance towards the pancreatic beta-cells in charge of insulin production and induces an immune response, mainly cell-mediated, associated to the production of autoantibodies, which leads to the self-destruction of beta-cells. The term “fumarate-related disease” encompasses all disorders or diseases that benefit from the treatment with fumarate.

Preferred examples of such diseases or disorders are auto-immune disorders, demyelinating diseases, transplant rejection and cancer. Preferred examples of such diseases and disorders are: Multiple Sclerosis (MS), psoriasis, Neuromyelitis optica (NMO), Rheumatoid Arthritis (RA), polyarthritis, asthma, atopic dermatitis, scleroderma, ulcerative colitis, juveline diabetes, thyreoiditis, Grave's disease, Systemic Lupus Erythromatosis (SLE), Sjogren syndrome, anemia perniciosa, chronic active hepatitis, transplant rejection and cancer. Preferred examples are MOG autoantigen-related diseases and disorders such as MS and NMO.

The term “demyelination” as used within the framework of demyelinating diseases or disorders herein refers to damaging and/or degradation of myelin sheaths that surround axons of neurons which has as a consequence the formation of lesions or plaques. Due to demyelination, the signal conduction along the affected nerves is impaired, and may cause neurological symptoms such as deficiencies in sensation, movement, cognition, and/or other neurological function. The concrete symptoms a patient suffering from a demyelinating disease will vary depending on the disease and disease progression state. These may include a blurred and/or double vision, ataxia, clonus, dysarthria, fatigue, clumsiness, hand paralysis, hemiparesis, genital anaesthesia, incoordination, paresthesias, ocular paralysis, impaired muscle coordination, muscle weakness, loss of sensation, impaired vision, neurological symptoms, unsteady way of walking (gait), spastic paraparesis, incontinence, hearing problems, speech problems, and others. Demyelinating diseases may be stratified into central nervous system demyelinating diseases and peripheral nervous system. Alternatively, demyelinating diseases may be classified according to the cause of demyelination: destruction of myelin (demyelinating myelinoclastic), or abnormal and degenerative myelin (dysmyelinating leukodystrophic). MS is considered in the art a demyelinating disorder of the central nervous system (Lubetzki and Stankoff. (2014). Handb Clin Neurol. 122, 89-99). Other specific but non-limiting examples of such demyelinating diseases and disorders include: neuromyelitis optica (NMO), acute inflammatory demyelinating polyneuropathy (AIDP), Chronic inflammatory demyelinating polyneuropathy (CIDP), acute transverse myelitis, progressive multifocal leucoencephalopathy (PML), acute disseminated encephalomyelitis (ADEM) or other hereditary demyelinating disorders.

The term “Multiple Sclerosis”, abbreviated herein and in the art as “MS”, indicates an autoimmune disorder affecting the central nervous system. MS is considered the most common non-traumatic disabling disease in young adults (Dobson and Giovannoni, (2019) Eur. J. Neurol. 26(1), 27-40), and the most common autoimmune disorder affecting the central nervous system (Berer and Krishnamoorthy (2014) FEBS Lett. 588(22), 4207-4213). MS may manifest itself in a subject by a large number of different symptoms ranging from physical over mental to psychiatric problems. Typical symptoms include blurred or double vision, muscle weakness, blindness in one eye, and difficulties in coordination and sensation. In most cases, MS may be viewed as a two-stage disease, with early inflammation responsible for relapsing-remitting disease and delayed neurodegeneration causing non-relapsing progression, i.e. secondary and primary progressive MS. Although progress is being made in the field, a conclusive underlying cause of the disease remains hitherto elusive and over 150 single nucleotide polymorphisms have been associated with MS susceptibility (International Multiple Sclerosis Genetics Consortium Nat Genet. (2013). 45(11):1353-60). Vitamin D deficiency, smoking, ultraviolet B (UVB) exposure, childhood obesity and infection by Epstein-Barr virus have been reported to contribute to disease development (Ascherio (2013) Expert Rev Neurother. 13(12 Suppl), 3-9).

Hence, MS can be regarded as a single disease existing within a spectrum extending from relapsing (wherein inflammation is the dominant feature) to progressive (neurodegeneration dominant). Therefore it is evident that the term Multiple sclerosis as used herein encompasses any type of Multiple Sclerosis belonging to any kind of disease course classification. In particular the invention is envisaged to be a potent treatment strategy patient diagnosed with, or suspected of having clinically Isolated Syndrome (CIS), relapse-remitting MS (RRMS), secondary progressive MS (SPMS), primary progressive MS (PPMS), and even MS-suspected radiology isolated syndrome (RIS). While strictly not considered a disease course of MS, RIS is used to classify subjects showing abnormalities on the Magnetic Resonance Imaging (MRI) of brain and/or spinal cord that correspond to MS lesions and cannot be prima facie explained by other diagnoses. CIS is a first episode (by definition lasting for over 24 hours) of neurologic symptoms caused by inflammation and demyelination in the central nervous system. In accordance with RIS, CIS classified subjects may or may not continue to develop MS, with subjects showing MS-like lesions on a brain MRI more likely to develop MS. RRMS is the most common disease course of MS with 85% of subjects having MS being diagnosed with RRMS. RRMS diagnosed patients are a preferred group of patients in view of the current invention. RRMS is characterized by attacks of new or increasing neurologic symptoms, alternatively worded relapses or exacerbations. In RRMS, said relapses are followed by periods or partial or complete remission of the symptoms, and no disease progression is experienced and/or observed in these periods of remission. RRMS may be further classified as active RRMS (relapses and/or evidence of new MRI activity), non-active RRMS, worsening RRMS (increasing disability over a specified period of time after a relapse, or not worsening RRMS. A portion of RRMS diagnosed subject will progress to the SPMS disease course, which is characterized by a progressive worsening of neurologic function, i.e. an accumulation of disability, over time. SPMS subclassifications can be made such as active (relapses and/or new MRI activity), not active, progressive (disease worsening over time), or non-progressive SPMS. Finally, PPMS is an MS disease course characterized by worsening of neurologic function and hence an accumulation of disability from the onset of symptoms, without early relapse or remission. Further PPMS subgroups can be formed such as active PPMS (occasional relapse and/or new MRI activity), non-active PPMS, progressive PPMS (evidence of disease worsening over time, regardless of new MRI activity) and non-progressive PPMS. In general, MS disease courses are characterized by substantial intersubject variability in terms of relapse and remission periods, both in severity (in case of relapse) and duration.

Several disease modifying therapies are available for MS, and therefore the current invention may be used as alternative treatment strategy, or in combination with these existing therapies. Non-limiting examples of active pharmaceutical ingredients include interferon beta-1a, interferon beta-1b, glatiramer acetate, glatiramer acetate, peginterferon beta-1a, teriflunomide, fingolimod, cladribine, siponimod, dimethyl fumarate, diroximel fumarate, ozanimod, alemtuzumab, mitoxantrone, ocrelizumab, and natalizumab. Alternatively, the invention may be used in combination with a treatment or medication aiming to relapse management, such as but not limited to methylprednisolone, prednisone, and adrenocorticotropic hormone(s) (ACTH). Further, the invention may be used in combination with a therapy aiming to alleviate specific symptoms. Non-limiting examples include medications aiming to improve or avoid symptoms selected from the group consisting of: bladder problems, bowel dysfunction, depression, dizziness, vertigo, emotional changes, fatigue, itching, pain, sexual problems, spasticity, tremors, and walking difficulties.

MS is characterized by three intertwined hallmark characteristics: 1) lesion formation in the central nervous system, 2) inflammation, and 3) degradation of myelin sheaths of neurons. Despite traditionally being considered a demyelinating disease of the central nervous system and white matter, more recently reports have surfaced that demyelination of the cortical and deep gray matter may exceed white matter demyelination (Kutzelnigg et al. (2005). Brain. 128(11), 2705-2712). Two main hypotheses have been postulated as to how MS is caused at the molecular level. The commonly accepted “outside-in hypothesis” is based on the activation of peripheral autoreactive effector CD4+ T cells which migrate to the central nervous system and initiate the disease process. Once in the central nervous system, said T cells are locally reactivated by APCs and recruit additional T cells and macrophages to establish inflammatory lesions. Noteworthy, MS lesions have been shown to contain CD8+ T cells predominantly found at the lesion edges, and CD4+ T cells found more central in the lesions. These cells are thought to cause demyelination, oligodendrocyte destruction, and axonal damage, leading to neurologic dysfunction. Additionally, immune-modulatory networks are triggered to limit inflammation and to initiate repair, which results in at least partial remyelination reflected by clinical remission. Nonetheless, without adequate treatment, further attacks often lead to progression of the disease.

MS onset is believed to originate well before the first clinical symptoms are detected, as evidenced by the typical occurrence of apparent older and inactive lesions on the MRI of patients. Due to advances in the development of diagnostic methods, MS can now be detected even before a clinical manifestation of the disease (i.e. pre-symptomatic MS). In the context of the invention, “treatment of MS” and similar expressions envisage treatment of, and treatment strategies for, both symptomatic and pre-symptomatic MS. In particular, when the immunogenic peptides and/or resulting cytolytic CD4+ T cells are used for treating a pre-symptomatic MS patient, the disease is halted at such an early stage that clinical manifestations may be partially, or even completely avoided. MS wherein the subject is not fully responsive to a treatment of interferon beta is also encompassed within the term “MS”.

The term “Neuromyelitis Optica” or “NMO” and “NMO Spectrum Disorder (NMOSD)”, also known as “Devic's disease”, refers to an autoimmune disorder in which white blood cells and antibodies primarily attack the optic nerves and the spinal cord, but may also attack the brain (reviewed in Wingerchuk 2006, Int MS J. 2006 May; 13(2):42-50). The damage to the optic nerves produces swelling and inflammation that cause pain and loss of vision; the damage to the spinal cord causes weakness or paralysis in the legs or arms, loss of sensation, and problems with bladder and bowel function. NMO is a relapsing-remitting disease. During a relapse, new damage to the optic nerves and/or spinal cord can lead to accumulating disability. Unlike MS, there is no progressive phase of this disease. Therefore, preventing attacks is critical to a good long-term outcome. In cases associated with anti-MOG antibodies, it is considered that anti-MOG antibodies may trigger an attack on the myelin sheath resulting in demyelination. The cause of NMO in the majority of cases is due to a specific attack on auto-antigens. Up to a third of subjects may be positive for auto-antibodies directed against a component of myelin called myelin oligodendrocyte glycoprotein (MOG). People with anti-MOG related NMO similarly have episodes of transverse myelitis and optic neuritis. Particularly envisaged within the framework of this application is NMO induced by MOG autoantigens and/or caused by anti-MOG antibodies.

The term “Rheumatoid Arthritis” or “RA” is an autoimmune, inflammatory disease that causes pain, swelling, stiffness, and loss of function in various joints (most commonly in the hands, wrists, and knees). The respective joint's lining becomes inflamed, leading to tissue damage, as well as chronic pain, unsteadiness, and deformity. There is generally a bilateral/symmetrical pattern of disease progression (e.g., both hands or both knees are affected). RA can also affect extra-articular sites, including the eyes, mouth, lungs, and heart. Patients can experience an acute worsening of their symptoms (called a flare) but with early intervention and appropriate treatment, symptoms can be ameliorated for a certain duration (reviewed by Sana Iqbal et al., 2019, US Pharm. 2019;44(1)(Specialty&Oncology suppl):8-11). The antigens attacked by the immune system and responsible for the disease are diverse but some examples are: GRP78, HSP60, 60 kDa chaperonin 2, Gelsolin, Chitinase-3-like protein 1, Cathepsin S, Serum albumin, and Cathepsin D.

The term “Psoriasis” refers to a chronic inflammatory skin disease with a strong genetic predisposition and autoimmune pathogenic traits. The worldwide prevalence is about 2%, but varies according to regions. It shows a lower prevalence in Asian and some African populations, and up to 11% in Caucasian and Scandinavian populations. The dermatologic manifestations of psoriasis are varied; psoriasis vulgaris is also called plaque-type psoriasis, and is the most prevalent type. The terms psoriasis and psoriasis vulgaris are used interchangeably in the scientific literature; nonetheless, there are important distinctions among the different clinical subtypes. Psoriasis Vulgaris (about 90% of psoriasis cases) is a chronic plaque-type psoriasis. The classical clinical manifestations are sharply demarcated, erythematous, pruritic plaques covered in silvery scales. The plaques can coalesce and cover large areas of skin. Common locations include the trunk, the extensor surfaces of the limbs, and the scalp. Other types are: Inverse Psoriasis, also called flexural psoriasis, affects intertriginous locations, and is characterized clinically by slightly erosive erythematous plaques and patches; Guttate Psoriasis, which is a variant with an acute onset of small erythematous plaques. It usually affects children or adolescents, and is often triggered by group-A streptococcal infections of tonsils. About one-third of patients with guttate psoriasis will develop plaque psoriasis throughout their adult life; Pustular psoriasis characterized by multiple, coalescing sterile pustules. Pustular psoriasis can be localized or generalized. Two distinct localized phenotypes have been described: psoriasis pustulosa palmoplantaris (PPP) and acrodermatitis continua of Hallopeau. Both of them affect the hands and feet; PPP is restricted to the palms and soles, and ACS is more distally located at the tips of fingers and toes, and affects the nail apparatus. Generalized pustular psoriasis presents with an acute and rapidly progressive course characterized by diffuse redness and subcorneal pustules, and is often accompanied by systemic symptoms. The hallmark of psoriasis is sustained inflammation that leads to uncontrolled keratinocyte proliferation and dysfunctional differentiation. The histology of the psoriatic plaque shows acanthosis (epidermal hyperplasia), which overlies inflammatory infiltrates composed of dermal dendritic cells, macrophages, T cells, and neutrophils. Neovascularization is also a prominent feature. The inflammatory pathways active in plaque psoriasis and the rest of the clinical variants overlap, but also display discrete differences that account for the different phenotype and treatment outcomes. (reviewed by Rendon and Schäkel, Int J Mol Sci. 2019 March; 20(6): 1475).

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence. The selection of the antigen whereon the epitope of the immunogenic or tolerogenic peptide as described herein is designed will depend on the fumarate-related disease.

The term “therapeutically effective amount” refers to an amount of the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the peptide of the invention or derivative thereof, which will lead to an improvement or restoration of the normal physiological situation. For instance, when used to therapeutically treat a mammal affected by an immune disorder, it is a daily amount peptide/kg body weight of the said mammal. Alternatively, where the administration is through gene-therapy, the amount of naked DNA or viral vectors is adjusted to ensure the local production of the relevant dosage of the peptide of the invention, derivative or homologue thereof.

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X is used for a position where any amino acid is accepted. Alternatives are indicated by listing the acceptable amino acids for a given position, between square brackets (‘[ ]’). For example: [CST] stands for an amino acid selected from Cys, Ser or Thr. Amino acids which are excluded as alternatives are indicated by listing them between curly brackets (‘{ }’). For example: {AM} stands for any amino acid except Ala and Met. The different elements in a motif are optionally separated from each other by a hyphen (-). Repetition of an identical element within a motif can be indicated by placing behind that element a numerical value or a numerical range between parentheses. For example X(2) corresponds to X-X or XX; X(2, 5) corresponds to 2, 3, 4 or 5 X amino acids, A(3) corresponds to A-A-A or AAA.

To distinguish between the amino acids X, those between H and C are called external amino acids X (single underlined in the above sequence), those within the redox motif are called internal amino acids X (double underlined in the above sequence).

X represents any amino acid, particularly an L-amino acid, more particularly one of the 20 naturally occurring L-amino acids.

A peptide, comprising a T cell epitope and a modified peptide motif sequence, having reducing activity is capable of generating a population of antigen-specific cytolytic CD4+ T cell towards antigen-presenting cells.

Accordingly, in its broadest sense, the invention relates to the use of peptides which comprise at least one T-cell epitope of an antigen (self or non-self) with a potential to trigger an immune reaction, and an “oxidoreductase”, “thioreductase” “thioredox”, or “redox” (all terms can be used interchangeable herein) sequence motif with a reducing activity on peptide disulfide bonds. The MHC class II T cell epitope and the modified redox motif sequence may be immediately adjacent to each other in the peptide or optionally separated by a one or more amino acids (so called linker sequence). Optionally the peptide additionally comprises an endosome targeting sequence and/or additional “flanking” sequences.

The peptides disclosed herein comprise an MHC class II T-cell epitope of an insulin antigen with a potential to trigger an immune reaction, and a modified redox motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

The (modified) redox motif may be positioned at the amino-terminus side of the T-cell epitope or at the carboxy-terminus of the T-cell epitope.

Peptide fragments with reducing activity are encountered in thioreductases which are small disulfide reducing enzymes including glutaredoxins, nucleoredoxins, thioredoxins and other thiol/disulfide oxidoreductases (Holmgren (2000) Antioxid. Redox Signal. 2, 811-820; Jacquot et al. (2002) Biochem. Pharm. 64, 1065-1069). They are multifunctional, ubiquitous and found in many prokaryotes and eukaryotes. They exert reducing activity for disulfide bonds on proteins (such as enzymes) through redox active cysteines within conserved active domain consensus sequences: CXXC [SEQ ID NO:18], CXXS [SEQ ID NO:23], CXXT [SEQ ID NO:24], SXXC [SEQ ID NO:21], TXXC [SEQ ID NO:22] (Fomenko et al. (2003) Biochemistry 42, 11214-11225; Fomenko et al. (2002) Prot. Science 11, 2285-2296), in which X stands for any amino acid. Such domains are also found in larger proteins such as protein disulfide isomerase (PDI) and phosphoinositide-specific phospholipase C.

The 4 amino acid redox motif as known from e.g. Fomenko and WO2008/017517 comprises a cysteine at position 1 and/or 4; thus the motif is either CXX[CST] [SEQ ID NO: 1] or [CST]XXC [SEQ ID NO:2]. Such a tetrapeptide sequence will be referred to as “the motif”. The motif in a peptide can be any of the alternatives CXXC [SEQ ID NO:137], SXXC [SEQ ID NO:138], TXXC [SEQ ID NO:139], CXXS [SEQ ID NO:140]or CXXT [SEQ ID NO:141]. In particular, peptides contain the sequence motif CXXC [SEQ ID NO:137].

As explained in detail further on, the peptides used in the present invention can be made by chemical synthesis, which allows the incorporation of non-natural amino acids. Accordingly, “C” in the above recited redox modified redox motifs represents either cysteine or another amino acid with a thiol group such as mercaptovaline, homocysteine or other natural or non-natural amino acids with a thiol function. In order to have reducing activity, the cysteines present in a modified redox motif should not occur as part of a cystine disulfide bridge. Nevertheless, a redox modified redox motif may comprise modified cysteines such as methylated cysteine, which is converted into cysteine with free thiol groups in vivo. X can be any of the 20 natural amino acids, including S, C, or T or can be a non-natural amino acid. In particular embodiments X is an amino acid with a small side chain such as Gly, Ala, Ser or Thr. In further particular embodiments, X is not an amino acid with a bulky side chain such as Trp. In further particular embodiments X is not Cysteine. In further particular embodiments at least one X in the modified redox motif is His. In other further particular embodiments at least one X in the modified redox is Pro.

Peptides may further comprise modifications to increase stability or solubility, such as modification of the N-terminal NH₂ group or the C terminal COOH group (e.g. modification of the COOH into a CONH₂ group).

The terms “oxidoreductase motif”, “thiol-oxidoreductase motif “, “thioreductase motif”, “thioredox motif” or “redox motif” are used herein as synonymous terms and refers to motifs involved in the transfer of electrons from one molecule (the reductant, also called the hydrogen or electron donor) to another (the oxidant, also called the hydrogen or electron acceptor).

The immunogenic peptides as defined herein comprise an oxidoreductase motif of the following general amino acid sequence: Zₘ-[CST]-Xₒ-C- (SEQ ID NO: 12 to 36) or Zₘ-C-Xₙ-[CST]- (SEQ ID NO: 37 to 61) as defined in aspect 2, is selected from the following amino acid motifs:
(a) Zₘ-[CST]-Xₙ-C- or Zₘ-C-Xₙ-[CST]- as defined in aspect 2, wherein n is 0, and

wherein wherein m is an integer selected from 0 to 3, wherein Z is any amino acid, preferably a basic amino acid selected from: H, K, R, and a non-natural basic amino acid as defined herein, such as L-ornithine, preferably K or R, most preferably K.

In preferred embodiments of motif (a), m is 1 or 2, and Z is a basic amino acid selected from: H, K, R, and a non-natural basic amino acid as defined herein, such as L-ornithine, preferably K or R, most preferably K.

Particularly preferred but non-limiting examples of such motifs are CC, KCC, KKCC (SEQ ID NO: 142), RCC, RKCC (SEQ ID NO: 143), KRCC (SEQ ID NO: 144), or RRCC (SEQ ID NO: 145).
(b) Zₘ-[CST]-Xₙ-C- or Zₘ-C-Xₙ-[CST]- as defined in aspect 2, wherein n is 1,

wherein X is any amino acid, preferably a basic amino acid selected from: H, K, R, and a non-natural basic amino acid such as L-ornithine, preferably K or R, most preferably K,

wherein m is an integer selected from 0 to 3,

wherein Z is any amino acid, preferably a basic amino acid selected from: H, K, R, and a non-natural basic amino acid as defined herein, such as L-ornithine, preferably K or R, most preferably K.

In preferred embodiments of motif (b), m is 1 or 2, and Z is a basic amino acid selected from: H, K, R, and a non-natural basic amino acid as defined herein, such as L-ornithine, preferably K or R, most preferably K.

Particularly preferred but non-limiting examples of such motifs are CRC, CKC, KCXC (SEQ ID NO: 146), KKCXC (SEQ ID NO: 147), RCXC (SEQ ID NO: 148), RRCXC (SEQ ID NO: 149), RKCXC (SEQ ID NO: 150), KRCXC (SEQ ID NO: 151), KCKC (SEQ ID NO: 152), KKCKC (SEQ ID NO: 153), KCRC (SEQ ID NO: 154), KKCRC (SEQ ID NO: 155), RCRC (SEQ ID NO: 156), RRCRC (SEQ ID NO: 157), RKCKC (SEQ ID NO: 158), or KRCKC (SEQ ID NO: 159).
(c) Zₘ-[CST]-Xₙ-C- or Zₘ-C-Xₙ-[CST]- as defined in aspect 2, wherein n is 2, thereby creating an internal X¹X² amino acid couple within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2.

In preferred embodiments, m is 1 and Z is a basic amino acid selected from: H, K, or R, or a non-natural basic amino acid as defined herein, such as L-ornithine, preferably K or R, most preferably K.

In preferred embodiments X¹ and X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a specific embodiment, at least one of X¹or X² in said motif is a basic amino acid selected from: H, K, or R, or a non-natural basic amino acid as defined herein, such as L-ornithine. In another specific embodiment, at least one of X¹or X² in said motif is P or Y. Specific examples of the internal X¹X2 amino acid couple within the oxidoreductase motif: PY, HY, KY, RY, PH, PK, PR, HG, KG, RG, HH, HK, HR, GP, HP, KP, RP, GH, GK, GR, GH, KH, and RH.

Particularly preferred motifs of this type are HCPYC, KCPYC, RCPYC, HCGHC, KCGHC, and RCGHC (corresponding to SEQ ID NO: 160 to 165).
(d) Zₘ-[CST]-Xₙ-C- or Zₘ-C-Xₙ-[CST]- as defined in aspect 2, wherein n is 3, thereby creating an internal X¹X2X³ amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2.

In some embodiments, X¹, X², and X³, each individually can be any amino acid selected from the group consisting of: G, A, V, L, I, M, F, W, P, S, T, C, Y, N, Q, D, E, K, R, and H, or non-natural amino acids. Preferably, X¹, X², and X³ in said motif is any amino acid except for C, S, or T. In a specific embodiment, at least one of X¹, X², or X³ in said motif is a basic amino acid selected from: H, K, or R, or a non-natural basic amino acid as defined herein, such as L-ornithine.

Specific examples of the internal X¹X2X³ amino acid stretch within the oxidoreductase motif are: XPY, PXY, and PYX, wherein X can be any amino acid, preferably a basic amino acid such as K, R, or H, or a non-natural basic amino acid such as L-ornithine.

Non-limiting examples are:
KPY, RPY, HPY, GPY, APY, VPY, LPY, IPY, MPY, FPY, WPY, PPY, SPY, TPY, CPY, YPY, NPY, QPY, DPY, EPY, and KPY; or
PKY, PRY, PHY, PGY, PAY, PVY, PLY, PIY, PMY, PFY, PWY, PPY, PSY, PTY, PCY, PYY, PNY, PQY, PDY, PEY, and PLY; or
PYK, PYR, PYH, PYG, PYA, PYV, PYL, PYI, PYM, PYF, PYW, PYP, PYS, PYT, PYC, PYY, PYN, PYQ, PYD, PYE, and PYL;
XHG, HXG, and HGX, wherein X can be any amino acid, such as in:
KHG, RHG, HHG, GHG, AHG, VHG, LHG, IHG, MHG, FHG, WHG, PHG, SHG, THG, CHG, YHG, NHG, QHG, DHG, EHG, and KHG; or
HKG, HRG, HHG, HGG, HAG, HVG, HLG, HIG, HMG, HFG, HWG, HPG, HSG, HTG, HCG, HYG, HNG, HQG, HDG, HEG, and HLG; or
HGK, HGR, HGH, HGG, HGA, HGV, HGL, HGI, HGM, HGF, HGW, HGP, HGS, HGT, HGC, HGY, HGN, HGQ, HGD, HGE, and HGL;
XGP, GXP, and GPX, wherein X can be any amino acid, such as in: KGP, RGP, HGP, GGP, AGP, VGP, LGP, IGP, MGP, FGP, WGP, PGP, SGP, TGP, CGP, YGP, NGP, QGP, DGP, EGP, and KGP; or
GKP, GRP, GHP, GGP, GAP, GVP, GLP, GIP, GMP, GFP, GWP, GPP, GSP, GTP, GCP, GYP, GNP, GQP, GDP, GEP, and GLP; or
GPK, GPR, GPH, GPG, GPA, GPV, GPL, GPI, GPM, GPF, GPW, GPP, GPS, GPT, GPC, GPY, GPN, GPQ, GPD, GPE, and GPL;
XGH, GXH, and GHX, wherein X can be any amino acid, such as in:
KGH, RGH, HGH, GGH, AGH, VGH, LGH, IGH, MGH, FGH, WGH, PGH, SGH, TGH, CGH, YGH, NGH, QGH, DGH, EGH, and KGH; or
GKH, GRH, GHH, GGH, GAH, GVH, GLH, GIH, GMH, GFH, GWH, GPH, GSH, GTH, GCH, GYH, GNH, GQH, GDH, GEH, and GLH; or
GHK, GHR, GHH, GHG, GHA, GHV, GHL, GHI, GHM, GHF, GHW, GHP, GHS, GHT, GHC, GHY, GHN, GHQ, GHD, GHE, and GHL;
XGF, GXF, and GFX, wherein X can be any amino acid, such as in:
KGF, RGF, HGF, GGF, AGF, VGF, LGF, IGF, MGF, FGF, WGF, PGF, SGF, TGF, CGF, YGF, NGF, QGF, DGF, EGF, and KGF; or
GKF, GRF, GHF, GGF, GAF, GVF, GLF, GIF, GMF, GFF, GWF, GPF, GSF, GTF, GCF, GYF, GNF, GQF, GDF, GEF, and GLF; or
GFK, GFR, GFH, GFG, GFA, GFV, GFL, GFI, GFM, GFF, GFW, GFP, GFS, GFT, GFC, GFY, GFN, GFQ, GFD, GFE, and GFL;
XRL, RXL, and RLX, wherein X can be any amino acid, such as in:
KRL, RRL, HRL, GRL, ARL, VRL, LRL, IRL, MRL, FRL, WRL, PRL, SRL, TRL, CRL, YRL, NRL, QRLRL, DRL, ERL, and KRL; or
GKF, GRF, GHF, GGF, GAF, GVF, GLF, GIF, GMF, GFF, GWF, GPF, GSF, GTF, GCF, GYF, GNF, GQF, GDF, GEF, and GLF; or
RLK, RLR, RLH, RLG, RLA, RLV, RLL, RLI, RLM, RLF, RLW, RLP, RLS, RLT, RLC, RLY, RLN, RLQ, RLD, RLE, and RLL;
XHP, HXP, and HPX, wherein X can be any amino acid, such as in: KHP, RHP, HHP, GHP, AHP, VHP, LHP, IHP, MHP, FHP, WHP, PHP, SHP, THP, CHP, YHP, NHP, QHP, DHP, EHP, and KHP; or
HKP, HRP, HHP, HGP, HAF, HVF, HLF, HIF, HMF, HFF, HWF, HPF, HSF, HTF, HCF, HYP, HNF, HQF, HDF, HEF, and HLP; or
HPK, HPR, HPH, HPG, HPA, HPV, HPL, HPI, HPM, HPF, HPW, HPP, HPS, HPT, HPC, HPY, HPN, HPQ, HPD, HPE, and HPL;

Particularly preferred examples are: CRPYC, KCRPYC, KHCRPYC, RCRPYC, HCRPYC, CPRYC, KCPRYC, RCPRYC, HCPRYC, CPYRC, KCPYRC, RCPYRC, HCPYRC, CKPYC, KCKPYC, RCKPYC, HCKPYC, CPKYC, KCPKYC, RCPKYC, HCPKYC, CPYKC, KCPYKC, RCPYKC, and HCPYKC (corresponding to SEQ ID NO: 166 to 190).
(e) Zₘ-[CST]-Xₙ-C- or Zₘ-C-Xₙ-[CST]- as defined in aspect 2, wherein n is 4, thereby creating an internal X¹X2X³X⁴(SEQ ID NO: 64) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2. X¹, X², X³ and X⁴ each individually can be any amino acid selected from the group consisting of: G, A, V, L, I, M, F, W, P, S, T, C, Y, N, Q, D, E, K, R, and H, or non-natural amino acids as defined herein. Preferably, X¹, X², X³ and X⁴ in said motif is any amino acid except for C, S, or T. In a specific embodiment, at least one of X¹, X², X³ or X⁴ in said motif is a basic amino acid selected from: H, K, or R, or a non-natural basic amino acid as defined herein.

Specific examples are: LAVL (SEQ ID NO: 191), TVQA (SEQ ID NO: 192) or GAVH (SEQ ID NO: 193) and their variants such as: X¹AVL, LX²VL, LAX³L, or LAVX⁴; X¹VQA, TX²QA, TVX³A, or TVQX⁴; X¹AVH, GX²VH, GAX³H, or GAVX⁴ (corresponding to SEQ ID NO: 194 to 205); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.
(f) Zₘ-[CST]-Xₙ-C- or Zₘ-C-Xₙ-[CST]- as defined in aspect 2, wherein n is 5, thereby creating an internal X¹X2X³X4X⁵ (SEQ ID NO: 65) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2. X¹, X², X³, X⁴ and X⁵ each individually can be any amino acid selected from the group consisting of: G, A, V, L, I, M, F, W, P, S, T, C, Y, N, Q, D, E, K, R, and H, or non-natural amino acids. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid selected from: H, K, or R, or a non-natural basic amino acid as defined herein.

Specific examples are: PAFPL (SEQ ID NO: 123) or DQGGE (SEQ ID NO: 124) and their variants such as: XIAFPL, PX²FPL, PAX³PL, PAFX⁴L, or PAFPX⁵; X¹QGGE, DX²GGE, DQX³GE, DQGX⁴E, or DQGGX⁵ (corresponding to SEQ ID NO: 208 to 217), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural amino acids as defined herein.
(g) Zₘ-[CST]-Xₙ-C- or Zₘ-C-Xₙ-[CST]- as defined in aspect 2, wherein n is 6, thereby creating an internal X¹X2X³X4X⁵X⁶(SEQ ID NO: 66) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2. X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural amino acid. Preferably, X¹, X², X³, X⁴, X⁵ and X⁶ in said motif is any amino acid except for C, S, or T.

In a specific embodiment, at least one of X¹, X², X³ X4, X⁵ or X⁶ in said motif is a basic amino acid selected from: H, K, or R, or a non-natural basic amino acid as defined herein.

Specific examples are: DIADKY (SEQ ID NO: 218) or variants thereof such as: X¹IADKY, DX²ADKY, DIX³DKY, DIAX⁴KY, DIADX⁵Y, or DIADKX⁶ (corresponding to SEQ ID NO: 219 to 224), wherein X¹, X², X³, X⁴, X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein. (h) Zₘ-[CST]-Xₙ-C- or Zₘ-C-Xₙ-[CST]-, wherein n is 0 to 6 and wherein m is 0, and wherein one of the C or [CST] residues has been modified so as to carry an acetyl, methyl, ethyl or propionyl group, either on the N-terminal amide of the amino acid residue of the motif or on the C-terminal carboxy group (SEQ ID NO: 144 to 163).

In preferred embodiments of such a motif, n is 2, and m is 0, wherein the internal X¹X2, each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹or X² in said motif is a basic amino acid selected from: H, K, or R, or a non-natural basic amino acid as defined herein, such as L-ornithine. In another example of the motif, at least one of X¹or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Preferably said modification results in an N-terminal acetylation of the first cysteine in the motif (N-acetyl-cysteine).

The term “basic amino acid” refers to any amino acid that acts like a Bronsted-Lowry and Lewis base, and includes natural basic amino acids such as Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-p-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

The oxidoreductase motif is placed either immediately adjacent to the epitope sequence within the peptide of the invention, or is separated from the T or NKT cell epitope by a linker. More particularly, the linker comprises an amino acid sequence of between 0 and 7 amino acids, that is 0, 1, 2, 3, 4, 5, 6, 7, 8, 9 or 10 amino acids.

Most particularly, the linker comprises an amino acid sequence of between 0 and 4 amino acids, that is 0, 1, 2, 3, or 4 amino acids. Alternatively, a linker may comprise 5, 6, 7, 8, 9 or 10 amino acids.

Apart from a peptide linker other organic compounds can be used as linker to link the parts of the peptide to each other (e.g. the oxidoreductase motif to the T or NKT cell epitope sequence).

The peptides of the present invention can further comprise additional short amino acid sequences N or C-terminally of the (artificial) sequence comprising the T or NKT cell epitope and the oxidoreductase motif. Such an amino acid sequence is generally referred to herein as a “flanking sequence”. A flanking sequence can be positioned between the epitope and an endosomal targeting sequence and/or between the oxidoreductase motif and an endosomal targeting sequence. In further embodiments, not comprising an endosomal targeting sequence, a short amino acid sequence may be present N and/or C terminally of the oxidoreductase motif and/or epitope sequence in the peptide. More particularly a flanking sequence is a sequence of between 1 and 7 amino acids, most particularly a sequence of 2 amino acids.

In any one of the motif embodiments herein, if m is 0 and in case of an N-terminal oxidoreductase motif (the oxidoreductase motif is located at the N-terminal beginning of the immunogenic peptide), the first cysteine, threonine or serine of the motif can be chemically modified through N-acetylation, N-methylation, N-ethylation or N-propionylation.

In any one of the motif embodiments herein, if m is 0 and in case of a C-terminal oxidoreductase motif (the oxidoreductase motif is located at the C-terminal end of the immunogenic peptide), the last cysteine, threonine or serine of the motif can be chemically modified through C-terminal substitution by acetyl, methyl, ethyl or propionyl groups of it's C-terminal amide or acid groups.

In the peptides used in the present invention comprising a redox motif, the motif is located such that, when the epitope fits into the MHC groove, the motif remains outside of the MHC binding groove. The modified redox motif is placed either immediately adjacent to the epitope sequence within the peptide [in other words a linker sequence of zero amino acids between motif and epitope], or is separated from the T cell epitope by a linker comprising an amino acid sequence of 7 amino acids or less. More particularly, the linker comprises 1, 2, 3, 4,5, 6 or 7 amino acids. Specific embodiments are peptides with a 0, 12, 3 or 4 amino acid linker between epitope sequence and modified redox motif sequence. Preferably the linker comprises an amino acid sequence of 4 amino acids. In those peptides where the modified redox motif sequence is adjacent to the epitope sequence this is indicated as position P-4 to P-1 or P+1 to P+4 compared to the epitope sequence. Apart from a peptide linker, other organic compounds can be used as linker to link the parts of the peptide to each other (e.g. the modified redox motif sequence to the T cell epitope sequence).

The peptides used in the present invention can further comprise additional short amino acid sequences N or C-terminally of the sequence comprising the T cell epitope and the modified redox motif. Such an amino acid sequence is generally referred to herein as a “flanking sequence”. A flanking sequence can be positioned between the epitope and an endosomal targeting sequence and/or between the modified redox motif and an endosomal targeting sequence. In certain peptides, not comprising an endosomal targeting sequence, a short amino acid sequence may be present N and/or C terminally of the modified redox motif and/or epitope sequence in the peptide. More particularly a flanking sequence is a sequence of between 1 and 7 amino acids, most particularly a sequence of 2 amino acids.

The modified redox motif may be located N-terminal from the epitope.

In certain embodiments of the present invention, peptides used are provided comprising one epitope sequence and a modified redox motif sequence. In further particular embodiments, the modified redox motif occurs several times (1, 2, 3, 4 or even more times) in the peptide, for example as repeats of the modified redox motif which can be spaced from each other by one or more amino acids or as repeats which are immediately adjacent to each other. Alternatively, one or more modified redox motifs are provided at both the N and the C terminus of the T cell epitope sequence.

Other variations envisaged for the peptides of the present invention include peptides which contain repeats of a T cell epitope sequence wherein each epitope sequence is preceded and/or followed by the modified redox motif (e.g. repeats of “modified redox motif-epitope” or repeats of “modified redox motif-epitope-modified redox motif”). Herein the modified redox motifs can all have the same sequence but this is not obligatory. It is noted that repetitive sequences of peptides which comprise an epitope which in itself comprises the modified redox motif will also result in a sequence comprising both the ‘epitope’ and a “modified redox motif”. In such peptides, the modified redox motif within one epitope sequence functions as a modified redox motif outside a second epitope sequence.

Typically the peptides used in the present invention comprise only one T cell epitope. As described below a T cell epitope in a protein sequence can be identified by functional assays and/or one or more in silica prediction assays. The amino acids in a T cell epitope sequence are numbered according to their position in the binding groove of the MHC proteins. A T-cell epitope present within a peptide consist of between 8 and 25 amino acids, yet more particularly of between 8 and 16 amino acids, yet most particularly consists of 8, 9, 10, 11, 12, 13, 14, 15 or 16 amino acids.

In a more particular embodiment, the T cell epitope consists of a sequence of 9 amino acids. In a further particular embodiment, the T-cell epitope is an epitope, which is presented to T cells by MHC-class II molecules [MHC class II restricted T cell epitopes]. Typically T cell epitope sequence refers to the octapeptide or more specifically nonapeptide sequence which fits into the cleft of an MHC II protein.

The T cell epitope of the peptides of the present invention can correspond either to a natural epitope sequence of a protein or can be a modified version thereof, provided the modified T cell epitope retains its ability to bind within the MHC cleft, similar to the natural T cell epitope sequence. The modified T cell epitope can have the same binding affinity for the MHC protein as the natural epitope, but can also have a lowered affinity. In particular, the binding affinity of the modified peptide is no less than 10-fold less than the original peptide, more particularly no less than 5 times less. Peptides of the present invention have a stabilising effect on protein complexes. Accordingly, the stabilising effect of the peptide-MHC complex compensates for the lowered affinity of the modified epitope for the MHC molecule.

The sequence comprising the T cell epitope and the reducing compound within the peptide can be further linked to an amino acid sequence (or another organic compound) that facilitates uptake of the peptide into late endosomes for processing and presentation within MHC class II determinants. The late endosome targeting is mediated by signals present in the cytoplasmic tail of proteins and correspond to well-identified peptide motifs. The late endosome targeting sequences allow for processing and efficient presentation of the antigen-derived T cell epitope by MHC-class II molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

Accordingly, the present invention envisages the use of peptides of antigenic proteins and their use in eliciting specific immune reactions. These peptides can either correspond to fragments of proteins which comprise, within their sequence i.e. a reducing compound and a T cell epitope separated by at most 10, preferably 7 amino acids or less. Alternatively, and for most antigenic proteins, the peptides of the invention are generated by coupling a reducing compound, more particularly a reducing modified redox motif as described herein, N-terminally or C-terminally to a T cell epitope of the antigenic protein (either directly adjacent thereto or with a linker of at most 10, more particularly at most 7 amino acids). Moreover the T cell epitope sequence of the protein and/or the modified redox motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the peptides of the present invention can comprise a sequence which is ‘artificial’ or “naturally occurring”.

The peptides of the present invention can vary substantially in length. The length of the peptides can vary from 13 or 14 amino acids, i.e. consisting of an epitope of 8-9 amino acids, adjacent thereto the modified redox motif 5 amino acids with the histidine, up to 20, 25, 30, 40 or 50 amino acids. For example, a peptide may comprise an endosomal targeting sequence of 40 amino acids, a flanking sequence of about 2 amino acids, a motif as described herein of 5 amino acids, a linker of 4 amino acids and a T cell epitope peptide of 9 amino acids.

Accordingly, in particular embodiments, the complete peptide consists of between 13 amino acids up 20, 25, 30, 40, 50, 75 or 100 amino acids. More particularly, where the reducing compound is a modified redox motif as described herein, the length of the (artificial or natural) sequence comprising the epitope and modified redox motif optionally connected by a linker (referred to herein as “epitope-modified redox motif” sequence), without the endosomal targeting sequence, is critical. The “epitope-modified redox motif” more particularly has a length of 13, 14, 15, 16, 17, 18 or 19 amino acids. Such peptides of 13 or 14 to 19 amino acids can optionally be coupled to an endosomal targeting signal of which the size is less critical.

As detailed above, in particular embodiments, the peptides of the present invention comprise a reducing modified redox motif as described herein linked to a T cell epitope sequence.

In further particular embodiments, the peptides used in the invention are peptides comprising T cell epitopes which do not comprise an amino acid sequence with redox properties within their natural sequence.

However, in alternative embodiments, the T cell epitope may comprise any sequence of amino acids ensuring the binding of the epitope to the MHC cleft. Where an epitope of interest of an antigenic protein comprises a modified redox motif such as described herein within its epitope sequence, the immunogenic peptides according to the present invention comprise the sequence of a modified redox motif as described herein and/or of another reducing sequence coupled N- or C- terminally to the epitope sequence such that (contrary to the modified redox motif present within the epitope, which is buried within the cleft) the attached modified redox motif can ensure the reducing activity.

Accordingly the T cell epitope and motif are immediately adjacent or separated from each other and do not overlap. To assess the concept of “immediately adjacent” or “separated”, the 8 or 9 amino acid sequence which fits in the MHC cleft is determined and the distance between this octapeptide or nonapeptide with the redox motif tetrapeptide or modified redox motif pentapeptide including histidine is determined.

Generally, the peptides used in the present invention are not natural (thus no fragments of proteins as such) but artificial peptides which contain, in addition to a T cell epitope, a modified redox motif as described herein, whereby the modified redox motif is immediately separated from the T cell epitope by a linker consisting of up to seven, most particularly up to four or up to 2 amino acids.

It has been shown that upon administration (i.e. injection) to a mammal of a peptide disclosed herein (or a composition comprising such a peptide), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells.

In this way, the peptides or composition comprising the peptides described in the present invention, which contain an antigen-derived T cell epitope and, outside the epitope, a modified redox motif can be used for direct immunisation of mammals, including human beings. The invention thus provides the use of peptides disclosed herein or derivatives thereof, for use as a medicine. Accordingly, the present invention provides therapeutic methods which comprise administering one or more peptides disclosed herein to a patient in need thereof.

The present invention offers methods by which antigen-specific T cells endowed with cytolytic properties can be elicited by immunisation with small peptides. It has been found that peptides which contain (i) a sequence encoding a T cell epitope from an antigen and (ii) a consensus sequence with redox properties, and further optionally also comprising a sequence to facilitate the uptake of the peptide into late endosomes for efficient MHC-class II presentation, elicit suppressor T-cells.

The immunogenic properties of the disclosed peptides are of particular interest in the treatment and prevention of immune reactions.

Peptides described herein are used as medicament, more particularly used for the manufacture of a medicament for the prevention or treatment of an immune disorder in a mammal, more in particular in a human.

The present invention describes methods of treatment or prevention of an immune disorder of a mammal in need for such treatment or prevention, by using the peptides disclosed herein, homologues or derivatives thereof, the methods comprising the step of administering to said mammal suffering or at risk of an immune disorder a therapeutically effective amount of the peptides disclosed herein, homologues or derivatives thereof such as to reduce the symptoms of the immune disorder. The treatment of both humans and animals, such as, pets and farm animals is envisaged.

In an embodiment the mammal to be treated is a human. The immune disorders referred to above are in a particular embodiment selected from allergic diseases and autoimmune diseases.

The peptides for use in the invention or the pharmaceutical composition comprising such peptides as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide for use in the invention or the pharmaceutical composition comprising such is administered in a therapeutically effective dose. Exemplary but non-limiting dosage regimens are between 300 and 1500 μg, preferably between 300 and 600 μg or between 1200 and 1500 μg.

Exemplary dosages are: from 300 to 600 μg of said immunogenic peptide;
from 600 to 800 μg of said immunogenic peptide;
from 800 to 1000 μg of said immunogenic peptide;
from 1000 to 1200 μg of said immunogenic peptide; or
from 1200 to 1500 μg of said immunogenic peptide

More specific dosage schemes can be between 300 and 500 μg, or about 450 μg or can be between 1300 and 1500 μg or about 1350 μg.

Dosage regimen can comprise the administration in a single dose or in 2, 3, 4, 5, 6 or more doses, either simultaneously or consecutively.

Exemplary non-limiting administration schemes are the following:
A low dose scheme comprising the SC administration of between 300 and 500 μg, or of about 450 μg of peptide in one or in two separate consecutive administrations, said administrations being repeated 4 to 6 times, with an interval of about 2 to 3 weeks, such as of about 2 weeks or such as of about 10 to 20 days, of about 11 to 19 days, of about 12 to 17 days, of about 13 to 16 days, of about 14 to 15 days, or of about 14 days.
A high dose scheme comprising the SC administration of between 1300 and 1500 μg or of about 1350 μg of peptide in one or in two separate consecutive administrations, said administrations being repeated 4 to 6 times, with an interval of 1 to 3 weeks, such as of about 2 weeks or such as of about 10 to 20 days, of about 11 to 19 days, of about 12 to 17 days, of about 13 to 16 days, of about 14 to 15 days, or of about 14 days.

Each of these treatment schemes can advantageously include a boost injection with the same dose at around week 24 to 30, counted from the start of the treatment, such as at week 24, 25, 26, 27, 28, 29, or 30 counted from the start of the treatment.

The peptides for use in the present invention can also be used in diagnostic in vitro methods for detecting class II restricted CD4+ T cells in a sample. In this method a sample is contacted with a complex of an MHC class II molecule and a peptide disclosed herein. The CD4+ T cells detected by measuring the binding of the complex with cells in the sample, wherein the binding of the complex to a cell is indicative for the presence of CD4+ T cells in the sample.

The complex can be a fusion protein of the peptide and an MHC class II molecule.

Alternatively MHC molecules in the complex are tetramers. The complex can be provided as a soluble molecule or can be attached to a carrier.

Accordingly, in particular embodiments, the methods of treatment and prevention of the present invention comprise the administration of an immunogenic peptide as described herein, wherein the peptide comprise a T cell epitope of an antigenic protein which plays a role in the disease to be treated (for instance such as those described above). In further particular embodiments, the epitope used is a dominant epitope, combined with method of stratification or selection of those patients that are assumed to benefit the most of said treatment.

Peptides for use in accordance with the present invention can be prepared by synthesising a peptide wherein T cell epitope and modified redox motif will be separated by 0 to 5 amino acids. In certain embodiments the modified redox motif can be obtained by introducing 1, 2 or 3 mutations outside the epitope sequence, to preserve the sequence context as occurring in the protein. Typically amino-acids in P-2 and P-1, as well as in P+10 and P+11, with reference to the nonapeptide which are part of the natural sequence are preserved in the peptide sequence. These flanking residues generally stabilize the binding to MHC class II. In other embodiments the sequence N terminal or C terminal of the epitope will be unrelated to the sequence of the antigenic protein containing the T cell epitope sequence.

Thus based upon the above methods for designing a peptide, a peptide is generated by chemical peptide synthesis, recombinant expression methods or in more exceptional cases, proteolytic or chemical fragmentation of proteins.

Peptides as produced in the above methods can be tested for the presence of a T cell epitope in in vitro and in vivo methods, and can be tested for their reducing activity in in vitro assays. As a final quality control, the peptides can be tested in in vitro assays to verify whether the peptides can generate CD4+ T cells which are cytolytic via an apoptotic pathway for antigen presenting cells presenting the antigen which contains the epitope sequence which is also present in the peptide with the modified redox motif.

The peptides for use in the present invention can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains such as methylated cysteine.

Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. If appropriate, such protecting groups can be left on the peptide after synthesis. Peptides can be linked to each other to form longer peptides using a ligation strategy (chemoselective coupling of two unprotected peptide fragments) as originally described by Kent (Schnelzer & Kent (1992)/nt. J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS.

Alternatively, the peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide of interest (e.g. solubility, stability) are examined to determine whether the peptide is/would be suitable for use in therapeutic compositions. Typically this is optimised by adjusting the sequence of the peptide. Optionally, the peptide can be modified after synthesis (chemical modifications e.g. adding/deleting functional groups) using techniques known in the art.

T cell epitopes on their own are thought to trigger early events at the level of the T helper cell by binding to an appropriate HLA molecule on the surface of an antigen presenting cell and stimulating the relevant T cell subpopulation. These events lead to T cell proliferation, lymphokine secretion, local inflammatory reactions, the recruitment of additional immune cells to the site, and activation of the B cell cascade leading to production of antibodies. One isotype of these antibodies, IgE, is fundamentally important in the development of allergic symptoms and its production is influenced early in the cascade of events, at the level of the T helper cell, by the nature of the lymphokines secreted. A T cell epitope is the basic element or smallest unit of recognition by a T cell receptor where the epitope comprises amino acid residues essential to receptor recognition, which are contiguous in the amino acid sequence of the protein.

However, upon administration of the peptides with a T-cell epitope and a redox motif, the following events are believed to happen:

activation of antigen (i) specific T cells resulting from cognate interaction with the antigen-derived peptide presented by MHC-class II molecules;

the reductase sequence reduces T cell surface proteins, such as the CD4 molecule, the second domain of which contains a constrained disulfide bridge. This transduces a signal into T cells. Among a series of consequences related to increased oxidative pathway, important events are increased calcium influx and translocation of the NF-kB transcription factor to the nucleus. The latter results in increased transcription of IFN-gamma and granzymes, which allows cells to acquire cytolytic properties via an apoptosis-inducing mechanism; the cytolytic property affects cells presenting the peptide by a mechanism, which involves granzyme B secretion, and Fas-FasL interactions. Since the cell killing effect is obtained via an apoptotic pathway, cytolytic cells is a more appropriate term for these cells than cytotoxic cells. Destruction of the antigen-presenting target cells prevents activation of other T cells specific for epitopes located on the same antigen, or to an unrelated antigen that would be processed by the same antigen-presenting cell; an additional consequence of T cell activation is to suppress activation of bystander T cells by a cell-cell contact dependent mechanism. In such a case, T cells activated by an antigen presented by a different antigen- presenting cell is also suppressed provided both cytolytic and bystander T cells are in close proximity, namely activated on the surface of the same antigen-presenting cell.

The above-postulated mechanism of action is substantiated with experimental data disclosed in the above cited PCT application WO2008/017517.

The present invention provides methods for generating antigen-specific cytolytic CD4+ T cells either in vivo or in vitro and their use in treating patients that have been stratified or selected as benefiting the most of said treatment. Independently thereof, methods to discriminate cytolytic CD4+ T cells from other cell populations such as Foxp3+ Tregs based on characteristic expression data can be envisaged.

The present invention describes in vivo methods for the production of the antigen-specific CD4+ T cells that can be used for treatment in light of the present invention. A particular embodiment relates to the method for producing or isolating the CD4+ T cells by immunising animals (including humans) with the peptides as described herein and then isolating the CD4+ T cells from the immunised animals. The present invention describes in vitro methods for the production of antigen specific cytolytic CD4+ T cells towards APC. The present application also discloses methods for generating antigen specific cytolytic CD4+ T cells towards APC.

In one embodiment, methods are provided which comprise the isolation of peripheral blood cells, the stimulation of the cell population in vitro by an immunogenic peptide described herein and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides described herein to a subject, and collection of the cytolytic CD4+ T cells generated in vivo.

The antigen-specific cytolytic CD4+ T cells towards APC, obtainable by the methods disclosed herein are of particular interest for the administration to mammals for immunotherapy, in the prevention of allergic reactions and the treatment of auto-immune diseases. Both the use of allogenic and autogeneic cells are envisaged.

Cytolytic CD4+ T cells populations are obtained as described herein below.

Antigen-specific cytolytic CD4+ T cells as described herein can be used as a medicament, more particularly for use in adoptive cell therapy, more particularly in the treatment of acute allergic reactions and relapses of autoimmune diseases such as multiple sclerosis. Isolated cytolytic CD4+ T cells or cell populations, more particularly antigen-specific cytolytic CD4+ T cell populations generated as described are used for the manufacture of a medicament for the prevention or treatment of immune disorders. Methods of treatment by using the isolated or generated cytolytic CD4+ T cells are disclosed.

The peptides for use in the invention will, upon administration to a living animal, typically a human being, elicit specific T cells exerting a suppressive activity on bystander T cells.

In specific embodiments the cytolytic cell populations disclosed herein are characterised by the expression of FasL and/or Interferon gamma. In specific embodiments the cytolytic cell populations of the present invention are further characterised by the expression of GranzymeB.

This mechanism also implies and the experimental results show that the peptides of the invention, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class II molecules in the vicinity of T cells activated by peptides of the invention.

Isolated cell populations of the cell type having the characteristics described above, which, in addition are antigen-specific, i.e. capable of suppressing an antigen-specific immune response are disclosed.

The present invention provides the use of pharmaceutical compositions comprising one or more peptides according to the present invention, further comprising a pharmaceutically acceptable carrier. As detailed above, the present invention also relates to the compositions for use as a medicine or to methods of treating a mammal of an immune disorder by using the composition and to the use of the compositions for the manufacture of a medicament for the prevention or treatment of immune disorders, combined with method of stratification or selection of those patients that are assumed to benefit the most of said treatment. The pharmaceutical composition could for example be a vaccine suitable for treating or preventing immune disorders, especially airborne and foodborne allergy, as well as diseases of allergic origin. As an example described further herein of a pharmaceutical composition, a peptide according to the invention is adsorbed on an adjuvant suitable for administration to mammals, such as aluminium hydroxide (alum). Typically, the desired dosage as described herein, such as 50 μg to 1500 μg of the peptide, adsorbed on alum, are injected by the subcutaneous route on 3 occasions at an interval of 2 weeks. It should be obvious for those skilled in the art that other routes of administration are possible, including oral, intranasal or intramuscular. Also, the number of injections and the amount injected can vary depending on the conditions to be treated. Further, other adjuvants than alum can be used, provided they facilitate peptide presentation in MHC-class II presentation and T cell activation. Thus, while it is possible for the active ingredients to be administered alone, they typically are presented as pharmaceutical formulations. The formulations, both for veterinary and for human use, of the present invention comprise at least one active ingredient, as above described, together with one or more pharmaceutically acceptable carriers. The present disclosure relates to pharmaceutical compositions, comprising, as an active ingredient, one or more peptides described herein, in admixture with a pharmaceutically acceptable carrier. The pharmaceutical composition should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat immune disorders.

The term “pharmaceutically acceptable carrier” as used herein means any material or substance with which the active ingredient is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic peptide in the composition. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders. Suitable pharmaceutical carriers for use in the pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one- step or multi-steps procedure, with the selected carrier material and, where appropriate, the other additives such as surface-active agents. They may also be prepared by micronisation, for instance in view to obtain them in the form of microspheres usually having a diameter of about 1 to 10 pm, namely for the manufacture of microcapsules for controlled or sustained release of the active ingredients.

Suitable surface-active agents, also known as emulgent or emulsifier, to be used in the pharmaceutical compositions of the present invention are non- ionic, cationic and/or anionic materials having good emulsifying, dispersing and/or wetting properties. Suitable anionic surfactants include both water- soluble soaps and water-soluble synthetic surface-active agents. Suitable soaps are alkaline or alkaline-earth metal salts, unsubstituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl- ethanolamine, phosphatidylserine, phosphatidylglycerine, lysolecithin, cardio lipin, dioctanylphosphatidylcholine, dipalmitoylphoshatidylcholine and their mixtures.

Suitable non-ionic surfactants include polyethoxylated and poly propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol -polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbucw”, 2 d ed. (Hanser Verlag, Vienna, 1981) and “Encyclopaedia of Surfactants, (Chemical Publishing Co., New York, 1981). Peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti- oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily dose or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polyniethyl methacrylate and the other above-described polymers. Such methods include colloid drug delivery systems like lipophilic compositions, microspheres, microemulsions, nanoparticles, nanocapsules and so on. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Cytolytic CD4+ T cells as obtained as described herein, induce APC apoptosis after MHC-class II dependent cognate activation, affecting both dendritic and B cells, as demonstrated in vitro and in vivo, and (2) suppress bystander T cells by a contact-dependent mechanism in the absence of IL-10 and/or TGF-beta. Cytolytic CD4+ T cells can be distinguished from both natural and adaptive Tregs, as discussed in detail in WO2008/017517.

The present invention will now be illustrated by means of the following examples which are provided without any limiting intention. Furthermore, all references described herein are explicitly included herein by reference.

### EXAMPLES

### Example 1: Sample Size

Participants are allocated to treatment or placebo in a 1:1:1 ratio (Placebo: investigational medicinal product 450 μg: investigational medicinal product 1350 μg). The trial's Bayesian adaptive design is based on a linear longitudinal random effects model, with operating characteristics evaluated via simulation. The design uses 84 participants (28:28:28) to achieve at least 80% power to detect a change of 0.2 nmol/L in the log-transformed Dried Blood Spots (DBS)C-peptide level between at least one treatment arm and placebo at 48 weeks (last planned visit).

### Example 2: Study Design

This is a multi-centre, dose comparison, randomized, double-blind, placebo-controlled study in patients with type 1 diabetes (T1D) within maximum 9 weeks of diagnosis (defined as the day of first insulin injection) at screening and within a maximum of 12 weeks from diagnosis to randomization.

### Main Study

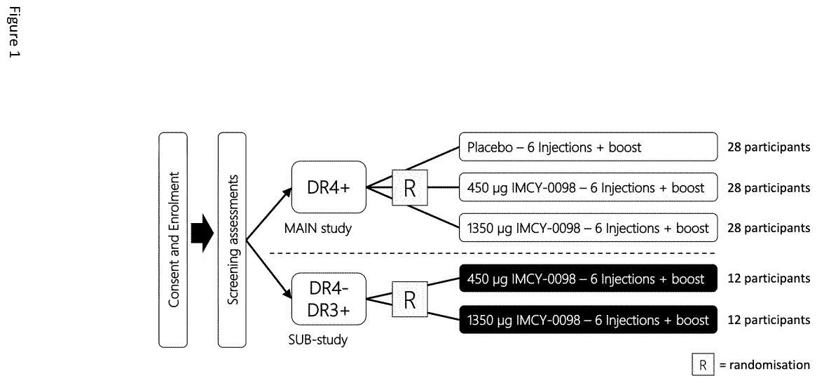

84 HLA DR4+ adult participants are randomly assigned (1:1:1) to one of the three treatment arms: placebo, investigational medicinal product (IMP) 450 μg and IMP 1350 μg (=the main study). All participants receive 6 administrations of IMP or placebo with a 2-week interval between each administration. At 24 weeks participants receive a boost injection. While treated participants receive a boost administration equivalent to the dose they have been randomized to, placebo participants receive a placebo boost. The participants were followed up to 48 weeks (FIG. 1).

### Substudy

Up to 24 HLA DR4 negative but HLA DR3+ adult participants are also included in a purely mechanistic substudy to study their immune response. Participants are randomly assigned to IMP 450 μg or IMP 1350 μg (=the substudy). Safety data are also collected and added to the full safety data set of the study (FIG. 1).

For each participant, including those included in the sub-study, the study comprises a total of 11 visits occurring for approximately 52 weeks (from screening visit to the last planned visit).

### Inclusion Criteria:

(1) Have given written informed consent.
(2) Participants aged 18 years and <45 years at the time of consent.
(3) Must have a diagnosis of T1D within maximum 9 weeks at screening (date of the first insulin injection).
(4) Must have at least one or more diabetes-related autoantibodies present at screening (GAD65, IA-2, or ZnT8).
(5) Must have random C-peptide levels 200 pmol/L measured at screening.
(6) Must be HLA DR4 positive for the main study.
   a. Up to 24 participants with an HLA DR4 negative status but HLA DR3 positive will be eligible for the substudy.
(7) Must be willing to comply with intensive diabetes management.
(8) Be treated with insulin therapy in accordance with the local standard of care.
(9) Males with reproductive potential must agree to use adequate contraception up to 90 days after the completion of the last treatment. This includes:
   Barrier contraception (condom and spermicide) or
   True abstinence (where this is in accordance with the participants preferred and usual lifestyle).
(10) All females must have a negative serum pregnancy test at screening. Women sexually active and of childbearing potential must agree to use a highly effective contraception method from screening up to 90 days after last treatment with the investigational product.

### Exclusion Criteria:

(1) Clinically significant abnormal full blood count (FBC), renal function or liver function at screening, including
   a. Be immunodeficient or have clinically significant chronic lymphopenia: Leukopenia (<3,000 leukocytes/μL), neutropenia (<1,500 neutrophils/μL), lymphopenia (<800 lymphocytes/μL), or thrombocytopenia (<100,000 platelets/μL).
   b. Evidence of renal dysfunction with creatinine greater than 1.5 times the upper limit of normal.
   c. Evidence of liver dysfunction with aspartate aminotransferase (AST) or alanine transaminase (ALT) greater than 3 times the upper limits of normal. Participants with elevated unconjugated bilirubin (Gilbert's syndrome) are eligible if bilirubin is 3 times the upper limits of normal and hepatic enzymes and function are otherwise normal (AST/ALT/Alkaline phosphatase within ULN), and there is no evidence of hemolysis.
(2) Have signs or symptoms of serious active infection requiring IV antibiotics and/or hospitalization at study entry.
(3) Have signs or symptoms of active COVID infection or a positive COVID PCR test during the screening period (refer to section 7.5 for further details).
(4) Have received any live, attenuated vaccine within 3 months prior to the first planned administration of the study product (which includes, but is not limited to: oral poliomyelitis vaccine, measles-mumps-rubella vaccine, yellow fever vaccine, Japanese encephalitis vaccine, dengue vaccine, rotavirus vaccine, varicella vaccine, live attenuated zoster vaccine, Bacillus Calmette-Gu6rin [BCG] vaccine, oral typhoid vaccine).
(5) Be currently pregnant or lactating, or anticipate getting pregnant until at least 24 weeks after last study drug administration.
(6) Require the use of immunosuppressive agents, including chronic use of systemic steroids. Topical, inhalational or intranasal corticosteroids are allowed.
(7) Have evidence of current or past human immunodeficiency virus (HIV), Hepatitis B or Hepatitis C infection.
(8) Presence of any uncontrolled disease (including uncontrolled autoimmune disease) or abnormal clinical laboratory results that may interfere with study conduct as judged by the investigator.
(9) History of, or current malignancy (except excised basal cell skin cancer).
(10)Current or ongoing use of non-insulin pharmaceuticals that affect glycaemic control within 7 days prior to screening visit.
(11) Active participation in another T1D treatment study or any investigational intervention study in the previous 30 days.
(12) Known hypersensitivity to any component of the drug product.
(13) CRO or Sponsor employees or employees under the direct supervision of the Investigator and/or involved directly in the study.

### Investigational Medicinal Product and Dosage

The investigational medicinal product (IMP) consists of a small synthetic peptide (20 amino acids) comprising a known human epitope of proinsulin (epitope C20-A1 LALEGSLQK, SEQ ID NO: 3) flanked with an oxidoreductase motif. The IMP has the following sequence: HCPYCSLQPLALEGSLQKRG (SED. ID NO: 73).

The IMP is presented in the form of a freeze-dried powder and solvent for subcutaneous (SC) administration. The solvent includes the adjuvant aluminium hydroxide (alum) at a concentration of 900 μg/mL.

Placebo is provided as a freeze-dried sterile powder made of 10 mg of mannitol for reconstitution with the same diluent as for the IMP.

Treatment consists of six administrations (separated by 14 days) of the IMP or the placebo by SC route. Half of the dose to be administered concomitantly in two sites (both the upper arms, in the region of the lateral part of the arm, midway between the elbow and the shoulder).

The dose A consists of six SC administrations of 450 μg of the peptide in two separate injections of 225 μg each (500 μL each arm). An additional administration (boost) is given at week 24.

The dose B consists of six SC administrations of 1350 μg of the peptide in two separate injections of 675 μg each (500 μL each arm). An additional administration (boost) is given at week 24.

The Placebo arm consisted of six SC administrations according to the same scheme as Dose A and B and a placebo boost administration at week 24 to keep the blind.

### Patient's Journey

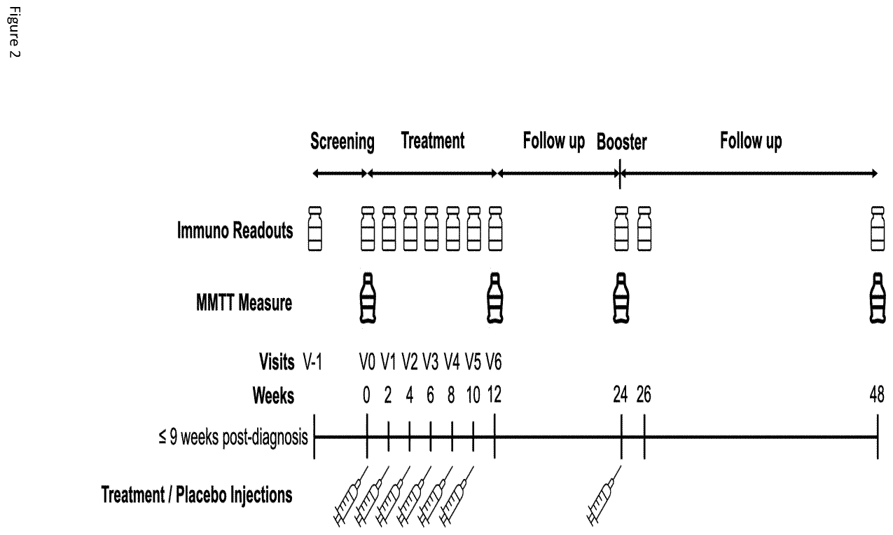

Patient's journey is as follows and is depicted in FIG. 2.

### Screening Assessment

Visit V-1 (within 9 Weeks from Diagnosis)

After signing the informed consent, the inclusion and exclusion criteria (including date of T1D diagnosis), demographic data, medical history and concomitant illness and the prior and current medication list are checked.

Investigator/designee checks vital signs, perform a complete physical examination and perform a 12-lead electrocardiogram (ECG).

Participants are swabbed for SARS-CoV2 virus presence (PCR).

A urine dipstick analysis is performed, and blood is collected for the following screening assessments:
C-peptide (random);
Autoantibodies;
HLA class I/class II genotype determination;
Immune cells (PBMC) isolation;
HbAlc;
Safety parameters: haematology (full blood count, including CD4/CD8 ratio), biochemistry (complete metabolic profile), virology;
Serum pregnancy test for all female participants.

### Treatment Period

### From Visit 0 (V0) to V7

At V0 (Day 0), V6 (week 12) and V7 (week 24), all participants have a 120 minutes mixed meal tolerance test (MMTT) with EnsurePlus for measuring C-peptide as a measurement of beta-cell response.

A stool sample is collected.

Blood sample is taken for HbA1c measurement.

A physical examination is performed.

At V7 a blood sample for autoantibodies detection is collected.

At each visit, Investigator/designee checked concomitant medication, vital signs, e-diary data and adverse event that occurred since the last visit. A urine dipstick is performed. For all women, urine is collected to perform a pregnancy test.

A blood sample is taken for safety parameters and PBMC isolation Participants then receive, except at V6, the SC dose of IMP at 450 μg or 1350 μg or placebo and receive instructions on the reporting of listed adverse events in e-diary for 7 days (visit day included).

### Follow-Up Period and End of the Study (EoS)

### At V6 (Week 12), V8 (Week 26) and V9 (Week 48)

At each visit, Investigator/designee checks the concomitant medication, vital signs, e-diary data and adverse event that occurred since the last visit.

A complete physical examination is performed¹.

A stool sample is collected¹.

A urine sample is collected, and a dipstick analysis is performed.

Blood was collected for the following assessments:
Immune cells (PBMC) isolation
HbAlc¹
Safety parameters
MMTT¹
at V8
At V9, a blood sample for autoantibodies detection is collected.
At V6 and V9, an ECG is performed.
At EoS visit (V9), all female participants have a urine pregnancy test.

All participants are offered to join the ongoing observational study for an additional 12 months post the end of the trial which will involve 1 single visit at 24 months from diagnosis.

Based on results from interim analysis, this additional follow-up visit at 24 months may be included in this protocol through an amendment.

Participants enrolled in this trial already receive the appropriate standard of care (insulin therapy), and this care continues after the study.

### Home Collection

### From V0 to EoS Visit (V9, Week 48)

DBS are collected at home pre and 60 min post-consumption of EnsurePlus, twice monthly, for the full 48 weeks follow-up, for DBS C-peptide measurement (new DBS cards are distributed at each visit).
Participants receive Continuous Glucose Monitoring (CGM) device, Dexcom G6, at VO. They are requested to use it continuously until the end of the study (new sensors are distributed at each visit) with a requirement to use it at least during the predefined periods at V0, V6, V7, and V9.

An e-diary is completed by the participant as instructed and relevant with insulin regimen, listed injection site reactions, other AEs and concomitant medication.

### Example 3: Procedure for Immune Analysis and Results

These analyses are performed in a blinded manner and the following results are communicated: The specific characteristics of the immune signature generated by the treatment. This may include the following parameters (non-exhaustive): activation markers; phenotypic markers (e.g. memory markers); cytokines profile.

In particular, the study aims to evaluate and characterize the proinsulin epitope C20-A1-specific CD4+ T cells after treatment with IMP.

These immune analyses rely on 2 laboratory methods, namely flow cytometry (FACS) and single-cell transcriptomic analysis. Following a short in vitro stimulation of PBMCs with natural proinsulin epitope C20-A1, cells are labelled with a panel of activation and characterization markers to phenotype and sort them.

### Blood Collection

Patient blood samples collected at six time points (visit V-1 or the baseline which was prior to immunization, and visits V2 to V6 which were two weeks after 2, 3, 4, 5 or 6 injections respectively) was used to quantify CD4+ T cells responding to proinsulin epitope C20-A1 stimulation.

Subsequently, single-cell transcriptomic analysis can also be applied using the 10x technology to characterize each cell transcriptome and cluster them in different subpopulations.

### Flow Cytometry

Cells were stained in different combinations of fluorochrome-conjugated antibodies. The LIVE/DEAD™ Fixable Near-IR Dead Cell Stain Kit (Thermo Fisher) was used to determine the viability of cells. FlowJo (Treestar®) software was used for analysis.

### FACS Data Analysis

Fluorescent activated cell sorting (FACS) analysis was performed to quantify CD4+ T cells that respond to proinsulin epitope stimulation. The responding cells were identified as CD4+ T cells which have either an effector phenotype or a regulatory phenotype. In a proinsulin epitope C20-A1-stimulated sample, the “net % of responding CD4+ T cells” was calculated as the % of CD4+ T cells with an effector or regulatory phenotype in the stimulated sample minus the % of CD4+ T cells with effector or regulatory phenotype in the corresponding unstimulated sample. For statistical analysis, patients were separated into two groups - IMP and Placebo treated. At each time point a Mann-Whiteny-Wilcoxon rank sum test was performed to determine if there was a significant difference between the net % of responding CD4+ T cells between these two groups. To test whether the treatment has an effect on the net % of responding CD4+ T cells over time, a repeated measures ANOVA model was fitted to the data separately for Imotope and Placebo treated patients. Mauchly's test of sphericity was used to verify whether the assumption of sphericity was met in the repeated measures ANOVA analysis. In case the sphericity condition was not met, Greenhouse-Geisser and Huynd-Feldt procedures were used to correct the p-values and the more conservative of the two p-values was taken. All statistical analysis was carried out in the R statistical environment.

### Results

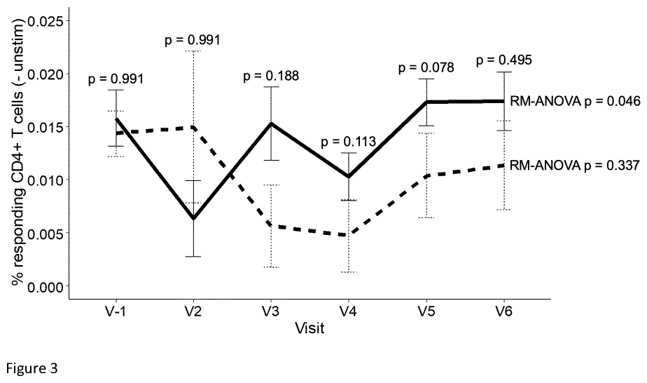

The data presented here was obtained from 24 patients, including 16 patients treated with the IMP (450 and 1350 μg) and 8 patients treated with Placebo. Statistical analysis of the net % of CD4+ T cells responding to proinsulin epitope C20-A1 stimulations (FIG. 3) showed that the IMP treatment had a statistically significant effect on the % of responding CD4+ T cells across time points (repeated measures ANOVA p-value of 0.046 after GG sphericity correction or 0.037 after HF sphericity correction), whereas the Placebo treatment did not show such effect (repeated measures ANOVA p-value of 0.337 after GG sphericity correction or 0.339 after HF sphericity correction). Higher presence of responding CD4+ T cells in IMP treated patients indicates that IMP-specific cytolytic CD4+ T cells could be induced. At individual time points, there was a trend of greater net % of responding CD4+ T cells in the IMP treated as compared to Placebo treated patients between visits 3 to 6 (which measured the impact of administrations 3 to 6). Statistical comparison of the % of responding CD4+ T cells between IMP and Placebo treated patients at individual time points using the nonparametric Mann-Whitney-Wilcoxon rank sum test did not reach statistical significance. However, the p-values dropped from V3 (p=0.188) to V4 (p=0.113) to V5 (p=0.078). This might indicate a trend of consistently improving response with time to 3, 4 and 5 administrations before the response plateaus after 6 administrations. Hence, a schedule of 5 or 6 administrations provides a sustained CD4+ T cell response that may be beneficial in controlling autoimmune diseases such as T1D.

1. A method of preventing or treating an auto-immune disorder, a demyelinating disorder, allograft or transplant rejection, a tumor or cancer, an infection with an intracellular pathogen, an immune response to a soluble allofactor, an immune response to an allergen exposure, or an immune response to a viral vector used for gene therapy or gene vaccination in a subject, comprising administering to the subject an immunogenic peptide, wherein said peptide comprises an oxidoreductase motif and, separated from this motif by 0 to 7 amino acids, an MHC class II T cell epitope sequence of an antigen involved in said disease or disorder,
wherein said oxidoreductase motif comprises the motif:
Zₘ-[CST]-Xₙ-C- or Zₘ-C-Xₙ-[CST]-,
wherein n is an integer selected from 2, 1, 0, 3, 4, 5, or 6, preferably 2, 1, 0, or 3. wherein m is for an integer from 0 to 2,
in which C stands for cysteine, S for serine, T for threonine, X for any amino acid and Z for any amino acid, preferably a basic amino acid,
wherein said immunogenic peptide is administered in at least 5 doses of from 300 to 1500 μg of said immunogenic peptide with an interval of about 12 days to about 28 days between two doses.

2. The method according to claim 1, wherein
(a) said administration is done through intramuscular or subcutaneous injection; or
(b) said immunogenic peptide is administered through intramuscular or subcutaneous injection of 6 doses of from 300 to 1500 μg of said immunogenic peptide with an interval of about 2 to 3 weeks between two doses.

3. The method according to claim 1,
wherein said immunogenic peptide is administered through intramuscular or subcutaneous injection of 6 doses of from 300 to 1500 μg of said immunogenic peptide with an interval of about 2 to 3 weeks between two doses.

4. The method according to claim 1, wherein
(a) the T cell epitope in said immunogenic peptide is not, or does not comprise an amino acid sequence selected from the group consisting of: MHC class II T cell epitopes FLRVPCWKI (SEQ ID NO: 4), and FLRVPSWKI (SEQ ID NO: 5), or NKT cell epitopes FLRVPCW (SEQ ID NO: 10), and FLRVPSW (SEQ ID NO: 11);
(b) the T cell epitope in said peptide is not a sequence derived from the MOG antigen amino acid sequence;
(c) said disease or disorder is not MS;
(d) said disease or disorder is not a disease that is known to be treated by fumarate, or is not a fumarate-related disease or disorder; or
(e) when said disease or disorder is MS, said treatment does not include the administration of a fumarate compound as defined herein.

5-8. (canceled)

9. The method according to claim 1, wherein each of said doses of from 300 to 1500 μg of said immunogenic peptide is administered with an interval of about 12 to about 16 days, or about 2 weeks between two doses.

10. The method according to claim 1, wherein each dose contains:
from 300 to 600 μg of said immunogenic peptide;
from 600 to 800 μg of said immunogenic peptide;
from 800 to 1000 μg of said immunogenic peptide;
from 1000 to 1200 μg of said immunogenic peptide; or
from 1200 to 1500 μg of said immunogenic peptide.

11. The method according to claim 1, wherein said dose contains 450 or 1350 μg of said immunogenic peptide.

12. The method according to claim 1, wherein said dose is administered 6 times, with an interval of about 12 to about 16 days, or about 2 weeks between doses.

13. The method according to claim 1, wherein a boost administration is performed of a dose of from 300 to 1500 μg of said immunogenic peptide
(a) at about week 22 to 30, counted from the start of the treatment;
(b) at about week 22 to 26 counted from the start of the treatment; or
(c) at about week 23 to 25 of the treatment, more preferably around week 24 of the treatment.

14. (canceled)

15. The method according to claim 13, wherein said boost contains:
from 300 to 600 μg of said immunogenic peptide;
from 600 to 800 μg of said immunogenic peptide;
from 800 to 1000 μg of said immunogenic peptide;
from 1000 to 1200 μg of said immunogenic peptide; or
from 1200 to 1500 μg of said immunogenic peptide.

16. The method according to claim 15, wherein said boost contains 450 or 1350 μg of said immunogenic peptide.

17. (canceled)

18. The method according to claim 1, wherein half of the dose is to be administered concomitantly in two sites (both upper arms, preferably in the region of the lateral part of the arms, more preferably midway between the elbow and the shoulder.

19. An in vitro method for analysing the response of a patient to the treatment of a disease or disorder selected from: an auto-immune disorder, a demyelinating disorder, allograft or transplant rejection, a tumor or cancer, an infection with an intracellular pathogen, an immune response to a soluble allofactor, an immune response to an allergen exposure, or an immune response to a viral vector used for gene therapy or gene vaccination in a subject, with an immunogenic peptide with a length of between 9 and 50 amino acids, said peptide comprising an oxidoreductase motif and, separated from this motif by 0 to 7 amino acids, an MHC class II T cell epitope sequence of an antigen involved in said disease or disorder, wherein said oxidoreductase motif comprises the motif:
Zₘ-[CST]-X₁-C- or Zₘ-C-Xₙ-[CST]-,
wherein n is an integer from 0 to 6, preferably 2, 1, 0, or 3.
wherein m is for an integer from 0 to 2,
in which C stands for cysteine, S for serine, T for threonine, X for any amino acid and Z for any amino acid, preferably a basic amino acid wherein said method comprises the analysis of samples taken from a patient being treated with said immunogenic peptide at the following time points:
Day 0 of the treatment,
In about week 11 to 13, such as in week 12 of the treatment,
In about week 23 to 25, such as in week 24 of the treatment, and
In about week 47 to 49, such as in week 48 of the treatment.

20. The in vitro method according to claim 19, wherein a sample of said patient is analysed about 8 to 10 weeks prior to the start of the treatment, preferably about 9 weeks prior to the start of said treatment.

21. The method according to claim 1, wherein said auto-immune disease is type-1-diabetes (T1D) or rheumatoid arthritis (RA).

22. The method according to claim 1, wherein the auto-immune disease is T1D and wherein the T-cell epitope in said peptide is an MHC class II T cell epitope from (pro-)insulin, preferably wherein the amino acid sequence of said epitope is defined by the amino acid sequence LALEGSLQK [SEQ ID NO: 3].

23. The method according to claim 22, wherein said patients are homozygous or heterozygous HLA type DR3 or DR4 positive.

24. The method according to claim 22, wherein said peptide comprises a sequence selected from the group consisting of:
[SEQ ID NO: 67]
Cxx[CST]SLQPLALEGSLQK,
[SEQ ID NO: 68]
[CST]xxCSLQPLALEGSLQK,
[SEQ ID NO: 69]
CxxCSLQPLALEGSLOK,
[SEQ ID NO: 70]
HCxx[CST]SLQPLALEGSLOK,
[SEQ ID NO:71]
H[CST]xxCSLQPLALEGSLOK,
[SEQ ID NO: 72]
HCxxCSLQPLALEGSLOK,
and
[SEQ ID NO: 73]
HCPYCSLQPLALEGSLQKRG.

25. The method according to claim 1, wherein
(a) the auto-immune disease is RA and wherein the T-cell epitope in said peptide is an MHC class II T cell or NKT cell epitope from an antigenic protein selected from the group comprising:
GRP78, HSP60, 60 kDa chaperonin 2, Gelsolin, Chitinase-3-like protein 1, Cathepsin S, Serum albumin, vinculin, and Cathepsin D;
(b) the auto-immune disease is Psoriasis and wherein said antigenic protein is selected from the group consisting of: ADAMTSL5, PLA2G4D, Keratin, such as Keratin 14 or Keratin 17, an antigen from Triticum aestivum, Pso p27, cathelicidin antimicrobial peptide, ceutrophil defensin 1 and LL37, preferably LL37;
(c) wherein the auto-immune disease is multiple sclerosis (MS) and wherein the T-cell epitope in said peptide is an MHC class II T cell or NKT cell epitope from an antigenic protein selected from the group comprising: Myelin Oligodendrocyte Glycoprotein (MOG), Myelin basic protein (MBP), Proteolipid protein (PLP), myelin-associated antigen (MAG), Oligodendrocyte-specific protein (OSP), myelin-associated oligodendrocyte basic protein (MOBP), 2′,3′-cyclic-nucleotide 3′-phosphodiesterase (CNPase), S100P protein and transaldolase H, preferably MOG: or
(d) the auto-immune disease is neuromyelitis optica (NMO) and wherein the T-cell epitope in said peptide is an MHC class II T cell or NKT cell epitope from Myelin Oligodendrocyte Glycoprotein (MOG),
(e) the auto-immune disease is multiple sclerosis (MS) or neuromyelitis optica (NMO) and the amino acid sequence of said epitope is defined by the amino acid sequence FLRVPSWKI (SEQ ID NO: 4); or
(e) the auto-immune disease is multiple sclerosis (MS) or neuromyelitis optica (NMO) and said immunogenic peptide has any one of the following sequences:
(SEQ ID NO: 85)
HCPYCVRYFLRVPSWKITLF,
(SEQ ID NO: 86)
HCPYCVRYFLRVPCWKITLF,
(SEQ ID NO: 87)
KHCPYCVRYFLRVPSWKITLFKK,
or
(SEQ ID NO: 88)
KHCPYCVRYFLRVPCWKITLFKK

26-27. (canceled)

28. The method according to claim 1, wherein
(a) said peptide is administered as a nucleic acid encoding said respective peptide;
(b) said peptide is administered as a nucleic acid encoding said respective peptide, wherein said nucleic acid encoding the immunogenic peptide is selected from isolated desoxyribonucleic acid (DNA), plasmid DNA (pDNA), coding DNA (cDNA), ribonucleic acid (RNA), messenger RNA (mRNA) or modified versions thereof, such as non-immunogenic mRNA comprising N(1)-methyl-pseudouridine (m1ψ); or
(c) said peptide is administered as a nucleic acid encoding said respective peptide, wherein said nucleic acid is part of an expression cassette, optionally incorporated in a (viral) vector or plasmid that is suitable to be used for gene-therapy or suitable to be present in the form of encapsulated or naked DNA or RNA.

29-32. (canceled)


SEQUENCE LISTING (248 sequences)

SEQ ID NO: 1 - protein, 4 aa, Artificial Sequence
  synthetic peptide
  PEPTIDE (2)..(3): X can be any amino acid
  PEPTIDE (4)..(4): X is C, S, or T
CXXX

SEQ ID NO: 2 - protein, 4 aa, Artificial Sequence
  synthetic peptide
  PEPTIDE (1)..(1): X is C, S, or T
  PEPTIDE (2)..(3): X can be any amino acid
XXXC

SEQ ID NO: 3 - protein, 9 aa, Homo Sapiens
LALEGSLQK

SEQ ID NO: 4 - protein, 9 aa, Homo Sapiens
FLRVPCWKI

SEQ ID NO: 5 - protein, 9 aa, Artificial Sequence
  synthetic peptide
FLRVPSWKI

SEQ ID NO: 6 - protein, 14 aa, Artificial Sequence
  synthetic peptide
  PEPTIDE (2)..(3): X can be any amino acid
  PEPTIDE (5)..(5): X can be any amino acid
  PEPTIDE (7)..(8): X can be any amino acid
  PEPTIDE (10)..(10): X can be any amino acid
  PEPTIDE (12)..(13): X can be any amino acid
CXXCXCXXCXCXXC

SEQ ID NO: 7 - protein, 12 aa, Artificial Sequence
  synthetic peptide
  PEPTIDE (2)..(3): X can be any amino acid
  PEPTIDE (6)..(7): X can be any amino acid
  PEPTIDE (10)..(11): X can be any amino acid
CXXCCXXCCXXC

SEQ ID NO: 8 - protein, 10 aa, Artificial Sequence
  synthetic peptide
  PEPTIDE (2)..(3): X can be any amino acid
  PEPTIDE (5)..(6): X can be any amino acid
  PEPTIDE (8)..(9): X can be any amino acid
CXXCXXCXXC

SEQ ID NO: 9 - protein, 10 aa, Artificial Sequence
  synthetic peptide
  PEPTIDE (2)..(2): X can be any amino acid
  PEPTIDE (5)..(5): X can be any amino acid
  PEPTIDE (8)..(8): X can be any amino acid
CXCCXCCXCC

SEQ ID NO: 10 - protein, 7 aa, Artificial Sequence
  synthetic peptide
FLRVPCW

SEQ ID NO: 11 - protein, 7 aa, Artificial Sequence
  synthetic peptide
FLRVPSW

SEQ ID NO: 12 - protein, 4 aa, Artificial Sequence
  synthetic peptide
  PEPTIDE (1)..(1): X is C, S, or T
  PEPTIDE (2)..(3): X can by any amino acid
XXXC

SEQ ID NO: 13 - protein, 5 aa, Artificial Sequence
  synthetic peptide
  PEPTIDE (1)..(1): X is C, S, or T
  PEPTIDE (2)..(4): X can be any amino acid
XXXXC

SEQ ID NO: 14 - protein, 6 aa, Artificial Sequence
  synthetic peptide
  PEPTIDE (1)..(1): X is C, S, or T
  PEPTIDE (2)..(5): X can be any amino acid
XXXXXC

SEQ ID NO: 15 - protein, 7 aa, Artificial Sequence
  synthetic peptide
  PEPTIDE (1)..(1): X is C, S, or T
  PEPTIDE (2)..(6): X can be any amino acid
XXXXXXC

SEQ ID NO: 16 - protein, 8 aa, Artificial Sequence
  synthetic peptide
  PEPTIDE (1)..(1): X is C, S, or T
  PEPTIDE (2)..(7): X can be any amino acid
XXXXXXXC

SEQ ID NO: 17 - protein, 4 aa, Artificial Sequence
  synthetic peptide
  PEPTIDE (1)..(1): X can be any amino acid
  PEPTIDE (2)..(2): X is C, S, or T
  PEPTIDE (3)..(3): X can be any amino acid
XXXC

SEQ ID NO: 18 - protein, 5 aa, Artificial Sequence
  synthetic peptide
  PEPTIDE (1)..(1): X can be any amino acid
  PEPTIDE (2)..(2): X is C, S, or T
  PEPTIDE (3)..(4): X can be any amino acid
XXXXC

SEQ ID NO: 19 - protein, 6 aa, Artificial Sequence
  synthetic peptide
  PEPTIDE (1)..(1): X can be any amino acid
  PEPTIDE (2)..(2): X is C, S, or T
  PEPTIDE (3)..(5): X can be any amino acid
XXXXXC

SEQ ID NO: 20 - protein, 7 aa, Artificial Sequence
  synthetic peptide
  PEPTIDE (1)..(1): X can be any amino acid
  PEPTIDE (2)..(2): X is C, S, or T
  PEPTIDE (3)..(6): X can be any amino acid
XXXXXXC

SEQ ID NO: 21 - protein, 8 aa, Artificial Sequence
  synthetic peptide
  PEPTIDE (1)..(1): X can be any amino acid
  PEPTIDE (2)..(2): X is C, S, or T
  PEPTIDE (3)..(7): X can be any amino acid
XXXXXXXC

SEQ ID NO: 22 - protein, 9 aa, Artificial Sequence
  synthetic peptide
  PEPTIDE (1)..(1): X can be any amino acid
  PEPTIDE (2)..(2): X is C, S, or T
  PEPTIDE (3)..(8): X can be any amino acid
XXXXXXXXC

SEQ ID NO: 23 - protein, 4 aa, Artificial Sequence
  synthetic peptide
  PEPTIDE (1)..(2): X can be any amino acid
  PEPTIDE (3)..(3): X is C, S, or T
XXXC

SEQ ID NO: 24 - protein, 5 aa, Artificial Sequence
  synthetic peptide
  PEPTIDE (1)..(2): X can be any amino acid
  PEPTIDE (3)..(3): X is C, S, or T
  PEPTIDE (4)..(4): X can be any acid
XXXXC

SEQ ID NO: 25 - protein, 6 aa, Artificial Sequence
  synthetic peptide
  PEPTIDE (1)..(2): X can be any amino acid
  PEPTIDE (3)..(3): X is C, S, or T
  PEPTIDE (4)..(5): X can be any amino acid
XXXXXC

SEQ ID NO: 26 - protein, 7 aa, Artificial Sequence
  synthetic peptide
  PEPTIDE (1)..(2): X can be any amino acid
  PEPTIDE (3)..(3): X is C, S, or T
  PEPTIDE (4)..(6): X can be any amino acid
XXXXXXC

SEQ ID NO: 27 - protein, 8 aa, Artificial Sequence
  synthetic peptide
  PEPTIDE (1)..(2): X can be any amino acid
  PEPTIDE (3)..(3): X is C, S, or T
  PEPTIDE (4)..(7): X can be any amino acid
XXXXXXXC

SEQ ID NO: 28 - protein, 9 aa, Artificial Sequence
  synthetic peptide
  PEPTIDE (1)..(2): X can be any amino acid
  PEPTIDE (3)..(3): X is C, S, or T
  PEPTIDE (4)..(8): X can be any amino acid
XXXXXXXXC

SEQ ID NO: 29 - protein, 10 aa, Artificial Sequence
  synthetic peptide
  PEPTIDE (1)..(2): X can be any amino acid
  PEPTIDE (3)..(3): X is C, S, or T
  PEPTIDE (4)..(9): X can be any amino acid
XXXXXXXXXC

SEQ ID NO: 30 - protein, 5 aa, Artificial Sequence
  synthetic peptide
  PEPTIDE (1)..(3): X can be any amino acid
  PEPTIDE (4)..(4): X is C, S, or T
XXXXC

SEQ ID NO: 31 - protein, 6 aa, Artificial Sequence
  synthetic peptide
  PEPTIDE (1)..(3): X can be any amino acid
  PEPTIDE (4)..(4): X is C, S, or T
  PEPTIDE (5)..(5): X can be any amino acid
XXXXXC

SEQ ID NO: 32 - protein, 7 aa, Artificial Sequence
  synthetic peptide
  PEPTIDE (1)..(3): X can be any amino acid
  PEPTIDE (4)..(4): X is C, S, or T
  PEPTIDE (5)..(6): X can be any amino acid
XXXXXXC

SEQ ID NO: 33 - protein, 8 aa, Artificial Sequence
  synthetic peptide
  PEPTIDE (1)..(3): X can be any amino acid
  PEPTIDE (4)..(4): X is C, S, or T
  PEPTIDE (5)..(7): X can be any amino acid
XXXXXXXC

SEQ ID NO: 34 - protein, 9 aa, Artificial Sequence
  synthetic peptide
  PEPTIDE (1)..(3): X can be any amino acid
  PEPTIDE (4)..(4): X is C, S, or T
  PEPTIDE (5)..(8): X can be any amino acid
XXXXXXXXC

SEQ ID NO: 35 - protein, 10 aa, Artificial Sequence
  synthetic peptide
  PEPTIDE (1)..(3): X can be any amino acid
  PEPTIDE (4)..(4): X is C, S, or T
  PEPTIDE (5)..(9): X can be any amino acid
XXXXXXXXXC

SEQ ID NO: 36 - protein, 11 aa, Artificial Sequence
  synthetic peptide
  PEPTIDE (1)..(3): X can be any amino acid
  PEPTIDE (4)..(4): X is C, S, or T
  PEPTIDE (5)..(10): X can be any amino acid
XXXXXXXXXXC

SEQ ID NO: 37 - protein, 4 aa, Artificial Sequence
  synthetic peptide
  PEPTIDE (2)..(3): X can be any amino acid
  PEPTIDE (4)..(4): X is C, S, or T
CXXX

SEQ ID NO: 38 - protein, 5 aa, Artificial Sequence
  synthetic peptide
  PEPTIDE (2)..(4): X can be any amino acid
  PEPTIDE (5)..(5): X is C, S, or T
CXXXX

SEQ ID NO: 39 - protein, 6 aa, Artificial Sequence
  synthetic peptide
  PEPTIDE (2)..(5): X can be any amino acid
  PEPTIDE (6)..(6): X is C, S, or T
CXXXXX

SEQ ID NO: 40 - protein, 7 aa, Artificial Sequence
  synthetic peptide
  PEPTIDE (2)..(6): X can be any amino acid
  PEPTIDE (7)..(7): X is C, S, or T
CXXXXXX

SEQ ID NO: 41 - protein, 8 aa, Artificial Sequence
  synthetic peptide
  PEPTIDE (2)..(7): X can be any amino acid
  PEPTIDE (8)..(8): X is C, S, or T
CXXXXXXX

SEQ ID NO: 42 - protein, 4 aa, Artificial Sequence
  synthetic peptide
  PEPTIDE (1)..(1): X can be any amino acid
  PEPTIDE (3)..(3): X can be any amino acid
  PEPTIDE (4)..(4): X is C, S, or T
XCXX

SEQ ID NO: 43 - protein, 5 aa, Artificial Sequence
  synthetic peptide
  PEPTIDE (1)..(1): X can be any amino acid
  PEPTIDE (3)..(4): X can be any amino acid
  PEPTIDE (5)..(5): X is C, S, or T
XCXXX

SEQ ID NO: 44 - protein, 6 aa, Artificial Sequence
  synthetic peptide
  PEPTIDE (1)..(1): X can be any amino acid
  PEPTIDE (3)..(5): X can be any amino acid
  PEPTIDE (6)..(6): X is C, S, or T
XCXXXX

SEQ ID NO: 45 - protein, 7 aa, Artificial Sequence
  synthetic peptide
  PEPTIDE (1)..(1): X can be any amino acid
  PEPTIDE (3)..(6): X can be any amino acid
  PEPTIDE (7)..(7): X is C, S, or T
XCXXXXX

SEQ ID NO: 46 - protein, 8 aa, Artificial Sequence
  synthetic peptide
  PEPTIDE (1)..(1): X can be any amino acid
  PEPTIDE (3)..(7): X can be any amino acid
  PEPTIDE (8)..(8): X is C, S, or T
XCXXXXXX

SEQ ID NO: 47 - protein, 9 aa, Artificial Sequence
  synthetic peptide
  PEPTIDE (1)..(1): X can be any amino acid
  PEPTIDE (3)..(8): X can be any amino acid
  PEPTIDE (9)..(9): X is C, S, or T
XCXXXXXXX

SEQ ID NO: 48 - protein, 4 aa, Artificial Sequence
  synthetic peptide
  PEPTIDE (1)..(2): X can be any amino acid
  PEPTIDE (4)..(4): X is C, S, or T
XXCX

SEQ ID NO: 49 - protein, 5 aa, Artificial Sequence
  synthetic peptide
  PEPTIDE (1)..(2): X can be any amino acid
  PEPTIDE (4)..(4): X can be any amino acid
  PEPTIDE (5)..(5): X is C, S, or T
XXCXX

SEQ ID NO: 50 - protein, 6 aa, Artificial Sequence
  synthetic peptide
  PEPTIDE (1)..(2): X can be any amino acid
  PEPTIDE (4)..(5): X can be any amino acid
  PEPTIDE (6)..(6): X is C, S, or T
XXCXXX

SEQ ID NO: 51 - protein, 7 aa, Artificial Sequence
  synthetic peptide
  PEPTIDE (1)..(2): X can be any amino acid
  PEPTIDE (4)..(6): X can be any amino acid
  PEPTIDE (7)..(7): X is C, S, or T
XXCXXXX

SEQ ID NO: 52 - protein, 8 aa, Artificial Sequence
  synthetic peptide
  PEPTIDE (1)..(2): X can be any amino acid
  PEPTIDE (4)..(7): X can be any amino acid
  PEPTIDE (8)..(8): X is C, S, or T
XXCXXXXX

SEQ ID NO: 53 - protein, 9 aa, Artificial Sequence
  synthetic sequence
  PEPTIDE (1)..(2): X can be any amino acid
  PEPTIDE (4)..(8): X can be any amino acid
  PEPTIDE (9)..(9): X is C, S, or T
XXCXXXXXX

SEQ ID NO: 54 - protein, 10 aa, Artificial Sequence
  synthetic peptide
  PEPTIDE (1)..(2): X can be any amino acid
  PEPTIDE (4)..(9): X can be any amino acid
  PEPTIDE (10)..(10): X is C, S, or T
XXCXXXXXXX

SEQ ID NO: 55 - protein, 5 aa, Artificial Sequence
  synthetic peptide
  PEPTIDE (1)..(3): X can be any amino acid
  PEPTIDE (5)..(5): X is C, S, or T
XXXCX

SEQ ID NO: 56 - protein, 6 aa, Artificial Sequence
  synthetic peptide
  PEPTIDE (1)..(3): X can be any amino acid
  PEPTIDE (5)..(5): X can be any amino acid
  PEPTIDE (6)..(6): X is C, S, or T
XXXCXX

SEQ ID NO: 57 - protein, 7 aa, Artificial Sequence
  synthetic peptide
  PEPTIDE (1)..(3): X can be any amino acid
  PEPTIDE (5)..(6): X can be any amino acid
  PEPTIDE (7)..(7): X is C, S, or T
XXXCXXX

SEQ ID NO: 58 - protein, 8 aa, Artificial Sequence
  synthetic peptide
  PEPTIDE (1)..(3): X can be any amino acid
  PEPTIDE (5)..(7): X can be any amino acid
  PEPTIDE (8)..(8): X is C, S, or T
XXXCXXXX

SEQ ID NO: 59 - protein, 9 aa, Artificial Sequence
  synthetic peptide
  PEPTIDE (1)..(3): X can be any amino acid
  PEPTIDE (5)..(8): X can be any amino acid
  PEPTIDE (9)..(9): X is C, S, or T
XXXCXXXXX

SEQ ID NO: 60 - protein, 10 aa, Artficial Sequence
  PEPTIDE (1)..(3): X can be any amino acid
  PEPTIDE (5)..(9): X can be any amino acid
  PEPTIDE (10)..(10): X is C, S, or T
XXXCXXXXXX

SEQ ID NO: 61 - protein, 11 aa, Artificial Sequence
  synthetic peptide
  PEPTIDE (1)..(3): X can be any amino acid
  PEPTIDE (5)..(10): X can be any amino acid
  PEPTIDE (11)..(11): X is C, S, or T
XXXCXXXXXXX

SEQ ID NO: 62 - protein, 7 aa, Artificial Sequence
  synthetic peptide
  PEPTIDE (1)..(1): X is F, W, H, or Y
  PEPTIDE (2)..(3): X can be any amino acid
  PEPTIDE (4)..(4): X is I, L, M, or V
  PEPTIDE (5)..(6): X can be any amino acid
  PEPTIDE (7)..(7): X is F, W, T, H, Y
XXXXXXX

SEQ ID NO: 63 - protein, 7 aa, Artificial Sequence
  synthetic peptide
  PEPTIDE (1)..(1): X is F, W
  PEPTIDE (2)..(3): X can be any amino acid
  PEPTIDE (4)..(4): X is I, L, M, or V
  PEPTIDE (5)..(6): X can be any amino acid
  PEPTIDE (7)..(7): X is F, W
XXXXXXX

SEQ ID NO: 64 - protein, 4 aa, Artificial Sequence
  synthetic peptide
  PEPTIDE (1)..(4): X can be any amino acid
XXXX

SEQ ID NO: 65 - protein, 5 aa, Artificial Sequence
  synthetic peptide
  PEPTIDE (1)..(5): X can be any amino acid
XXXXX

SEQ ID NO: 66 - protein, 6 aa, Artificial Sequence
  synthetic peptide
  PEPTIDE (1)..(6): X can be any amino acid
XXXXXX

SEQ ID NO: 67 - protein, 17 aa, Artificial Sequence
  synthetic peptide
  PEPTIDE (2)..(3): X can be any amino acid
  PEPTIDE (4)..(4): X is C or S or T
CXXXSLQPLALEGSLQK

SEQ ID NO: 68 - protein, 17 aa, Artificial Sequence
  synthetic peptide
  PEPTIDE (1)..(1): X is C, S or T
  PEPTIDE (2)..(3): X can be any amino acid
XXXCSLQPLALEGSLQK

SEQ ID NO: 69 - protein, 17 aa, Artificial Sequence
  synthetic peptide
  PEPTIDE (2)..(3): X can be any amino acid
CXXCSLQPLALEGSLQK

SEQ ID NO: 70 - protein, 18 aa, Artificial Sequence
  synthetic peptide
  PEPTIDE (3)..(4): X can be any amino acid
  PEPTIDE (5)..(5): X is C, S or T
HCXXXSLQPLALEGSLQK

SEQ ID NO: 71 - protein, 18 aa, Artificial Sequence
  synthetic peptide
  PEPTIDE (2)..(2): X is C, S or T
  PEPTIDE (3)..(4): X can be any amino acid
HXXXCSLQPLALEGSLQK

SEQ ID NO: 72 - protein, 18 aa, Artificial Sequence
  synthetic peptide
  PEPTIDE (3)..(4): X can be any amino acid
HCXXCSLQPLALEGSLQK

SEQ ID NO: 73 - protein, 20 aa, Artificial Sequence
  synthetic peptide
HCPYCSLQPLALEGSLQKRG

SEQ ID NO: 74 - protein, 218 aa, Artificial Sequence
  Homo sapiens Myelin-oligodendrocyte glycoprotein (MOG)
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 75 - protein, 21 aa, Artificial Sequence
  synthetic peptide
YRPPFSRVVHLYRNGKDQDGD

SEQ ID NO: 76 - protein, 15 aa, Artificial Sequence
  synthetic peptide
RPPFSRVVHLYRNGK

SEQ ID NO: 77 - protein, 15 aa, Artificial Sequence
  synthetic peptide
PFSRVVHLYRNGKDQ

SEQ ID NO: 78 - protein, 15 aa, Artificial Sequence
  synthetic peptide
FSRVVHLYRNGKDQD

SEQ ID NO: 79 - protein, 15 aa, Artificial Sequence
  synthetic peptide
SRVVHLYRNGKDQDG

SEQ ID NO: 80 - protein, 9 aa, Artificial Sequence
  synthetic peptide
VVHLYRNGK

SEQ ID NO: 81 - protein, 21 aa, Artificial Sequence
  synthetic peptide
MEVGWYRSPFSRVVHLYRNGK

SEQ ID NO: 82 - protein, 21 aa, Artificial Sequence
  synthetic peptide
MEVGWYRPPFSRVVHLYRNGK

SEQ ID NO: 83 - protein, 9 aa, Artificial Sequence
  synthetic peptide
YRSPFSRVV

SEQ ID NO: 84 - protein, 9 aa, Artificial Sequence
  synthetic peptide
YRPPFSRVV

SEQ ID NO: 85 - protein, 20 aa, Artificial Sequence
  synthetic peptide
HCPYCVRYFLRVPSWKITLF

SEQ ID NO: 86 - protein, 20 aa, Artificial Sequence
  synthetic peptide
HCPYCVRYFLRVPCWKITLF

SEQ ID NO: 87 - protein, 23 aa, Artificial Sequence
  synthetic peptide
KHCPYCVRYFLRVPSWKITLFKK

SEQ ID NO: 88 - protein, 23 aa, Artificial Sequence
  synthetic peptide
KHCPYCVRYFLRVPCWKITLFKK

SEQ ID NO: 89 - protein, 277 aa, Artificial Sequence
  Homo sapiens proteolipid protein (PLP)
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 90 - protein, 26 aa, Artificial Sequence
  synthetic peptide
HEALTGTEKLIETYFSKNYQDYEYLI

SEQ ID NO: 91 - protein, 28 aa, Artificial Sequence
  synthetic peptide
TWTTCQSIAFPSKTSASIGSLCADARMY

SEQ ID NO: 92 - protein, 28 aa, Artificial Sequence
  synthetic peptide
GVLPWNAFPGKVCGSNLLSICKTAEFQM

SEQ ID NO: 93 - protein, 19 aa, Artificial Sequence
  synthetic peptide
LTGTEKLIETYFSKNYQDY

SEQ ID NO: 94 - protein, 19 aa, Artificial Sequence
  synthetic peptide
WTTCQSIAFPSKTSASIGS

SEQ ID NO: 95 - protein, 15 aa, Artificial Sequence
  synthetic peptide
VLPWNAFPGKVCGSN

SEQ ID NO: 96 - protein, 15 aa, Artificial Sequence
  synthetic peptide
LTGTEKLIETYFSKN

SEQ ID NO: 97 - protein, 15 aa, Artificial Sequence
  synthetic peptide
TEKLIETYFSKNYQD

SEQ ID NO: 98 - protein, 15 aa, Artificial Sequence
  synthetic peptide
EKLIETYFSKNYQDY

SEQ ID NO: 99 - protein, 15 aa, Artificial Sequence
  synthetic peptide
WTTCQSIAFPSKTSA

SEQ ID NO: 100 - protein, 15 aa, Artificial Sequence
  synthetic peptide
TTCQSIAFPSKTSAS

SEQ ID NO: 101 - protein, 15 aa, Artificial Sequence
  synthetic peptide
TCQSIAFPSKTSASI

SEQ ID NO: 102 - protein, 15 aa, Artificial Sequence
  synthetic peptide
CQSIAFPSKTSASIG

SEQ ID NO: 103 - protein, 15 aa, Artificial Sequence
  synthetic peptide
QSIAFPSKTSASIGS

SEQ ID NO: 104 - protein, 15 aa, Artificial Sequence
  synthetic peptide
VLPWNAFPGKVCGSN

SEQ ID NO: 105 - protein, 26 aa, Artificial Sequence
  synthetic peptide
HEALTGTEKLIETYFSKNYQDYEYLI

SEQ ID NO: 106 - protein, 28 aa, Artificial Sequence
  synthetic peptide
TWTTCQSIAFPSKTSASIGSLCADARMY

SEQ ID NO: 107 - protein, 28 aa, Artificial Sequence
  synthetic peptide
GVLPWNAFPGKVCGSNLLSICKTAEFQM

SEQ ID NO: 108 - protein, 15 aa, Artificial Sequence
  synthetic peptide
PRHRDTGILDSIGRF

SEQ ID NO: 109 - protein, 17 aa, Artificial Sequence
  synthetic peptide
ENPVVHFFKNIVTPRTP

SEQ ID NO: 110 - protein, 15 aa, Artificial Sequence
  synthetic peptide
RASDYKSAHKGFKGV

SEQ ID NO: 111 - protein, 15 aa, Artificial Sequence
  synthetic peptide
GFKGVDAQGTLSKIF

SEQ ID NO: 112 - protein, 15 aa, Artificial Sequence
  synthetic peptide
LGGRDSRSGSPMARR

SEQ ID NO: 113 - protein, 20 aa, Artificial Sequence
  synthetic peptide
TQDENPVVHFFKNIVTPRTP

SEQ ID NO: 114 - protein, 15 aa, Artificial Sequence
  synthetic peptide
TQDENPVVHFFKNIV

SEQ ID NO: 115 - protein, 15 aa, Artificial Sequence
  synthetic peptide
QDENPVVHFFKNIVT

SEQ ID NO: 116 - protein, 15 aa, Artificial Sequence
  synthetic peptide
DENPVVHFFKNIVTP

SEQ ID NO: 117 - protein, 15 aa, Artificial Sequence
  synthetic peptide
ENPVVHFFKNIVTPR

SEQ ID NO: 118 - protein, 15 aa, Artificial Sequence
  synthetic peptide
NPVVHFFKNIVTPRT

SEQ ID NO: 119 - protein, 15 aa, Artificial Sequence
  synthetic peptide
PVVHFFKNIVTPRTP

SEQ ID NO: 120 - protein, 28 aa, Artificial Sequence
  synthetic peptide
ASDYKSAHKGFKGVDAQGTLSKIFKLGG

SEQ ID NO: 121 - protein, 15 aa, Artificial Sequence
  synthetic peptide
ASDYKSAHKGFKGVD

SEQ ID NO: 122 - protein, 15 aa, Artificial Sequence
  synthetic peptide
SDYKSAHKGFKGVDA

SEQ ID NO: 123 - protein, 15 aa, Artificial Sequence
  synthetic peptide
DYKSAHKGFKGVDAQ

SEQ ID NO: 124 - protein, 15 aa, Artificial Sequence
  synthetic peptide
YKSAHKGFKGVDAQG

SEQ ID NO: 125 - protein, 15 aa, Artificial Sequence
  synthetic peptide
KSAHKGFKGVDAQGT

SEQ ID NO: 126 - protein, 15 aa, Artificial Sequence
  synthetic peptide
SAHKGFKGVDAQGTL

SEQ ID NO: 127 - protein, 15 aa, Artificial Sequence
  synthetic peptide
AHKGFKGVDAQGTLS

SEQ ID NO: 128 - protein, 15 aa, Artificial Sequence
  synthetic peptide
HKGFKGVDAQGTLSK

SEQ ID NO: 129 - protein, 15 aa, Artificial Sequence
  synthetic peptide
KGFKGVDAQGTLSKI

SEQ ID NO: 130 - protein, 15 aa, Artificial Sequence
  synthetic peptide
GFKGVDAQGTLSKIF

SEQ ID NO: 131 - protein, 15 aa, Artificial Sequence
  synthetic peptide
FKGVDAQGTLSKIFK

SEQ ID NO: 132 - protein, 15 aa, Artificial Sequence
  synthetic peptide
KGVDAQGTLSKIFKL

SEQ ID NO: 133 - protein, 15 aa, Artificial Sequence
  synthetic peptide
GVDAQGTLSKIFKLG

SEQ ID NO: 134 - protein, 15 aa, Artificial Sequence
  synthetic peptide
VDAQGTLSKIFKLGG

SEQ ID NO: 135 - protein, 14 aa, Artificial Sequence
  synthetic peptide
LSRFSWGAEGQRPG

SEQ ID NO: 136 - protein, 171 aa, Artificial Sequence
  Homo sapiens MBP
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 137 - protein, 4 aa, Artificial Sequence
  synthetic peptide
  PEPTIDE (2)..(3): X can be any amino acid
CXXC

SEQ ID NO: 138 - protein, 4 aa, Artificial Sequence
  synthetic peptide
  PEPTIDE (2)..(3): X can be any amino acid
SXXC

SEQ ID NO: 139 - protein, 4 aa, Artificial Sequence
  synthetic peptide
  PEPTIDE (2)..(3): X can be any amino acid
TXXC

SEQ ID NO: 140 - protein, 4 aa, Artificial Sequence
  synthetic peptide
  PEPTIDE (2)..(3): X can be any amino acid
CXXS

SEQ ID NO: 141 - protein, 4 aa, Artificial Sequence
  synthetic peptide
  PEPTIDE (2)..(3): X can be any amino acid
CXXT

SEQ ID NO: 142 - protein, 4 aa, Artificial Sequence
  synthetic peptide
KKCC

SEQ ID NO: 143 - protein, 4 aa, Artificial Sequence
  synthetic peptide
RKCC

SEQ ID NO: 144 - protein, 4 aa, Artificial Sequence
  synthetic peptide
KRCC

SEQ ID NO: 145 - protein, 4 aa, Artificial Sequence
  synthetic peptide
RRCC

SEQ ID NO: 146 - protein, 4 aa, Artificial Sequence
  synthetic peptide
  PEPTIDE (3)..(3): X can be any amino acid
KCXC

SEQ ID NO: 147 - protein, 5 aa, Artificial Sequence
  synthetic peptide
  PEPTIDE (4)..(4): X can be any amino acid
KKCXC

SEQ ID NO: 148 - protein, 4 aa, Artificial Sequence
  synthetic peptide
  PEPTIDE (3)..(3): X can be any amino acid
RCXC

SEQ ID NO: 149 - protein, 5 aa, Artificial Sequence
  synthetic peptide
  PEPTIDE (4)..(4): X can be any amino acid
RRCXC

SEQ ID NO: 150 - protein, 5 aa, Artificial Sequence
  synthetic peptide
  PEPTIDE (4)..(4): X can be any amino acid
RKCXC

SEQ ID NO: 151 - protein, 5 aa, Artificial Sequence
  synthetic peptide
  PEPTIDE (4)..(4): X can be any amino acid
KRCXC

SEQ ID NO: 152 - protein, 4 aa, Artificial Sequence
  synthetic peptide
KCKC

SEQ ID NO: 153 - protein, 5 aa, Artificial Sequence
  synthetic peptide
KKCKC

SEQ ID NO: 154 - protein, 4 aa, Artificial Sequence
  synthetic peptide
KCRC

SEQ ID NO: 155 - protein, 5 aa, Artificial Sequence
  synthetic peptide
KKCRC

SEQ ID NO: 156 - protein, 4 aa, Artificial Sequence
  synthetic peptide
RCRC

SEQ ID NO: 157 - protein, 5 aa, Artificial Sequence
  synthetic peptide
RRCRC

SEQ ID NO: 158 - protein, 5 aa, Artificial Sequence
  synthetic peptide
RKCKC

SEQ ID NO: 159 - protein, 5 aa, Artificial Sequence
  synthetic peptide
KRCKC

SEQ ID NO: 160 - protein, 5 aa, Artificial Sequence
  synthetic peptide
HCPYC

SEQ ID NO: 161 - protein, 5 aa, Artificial Sequence
  synthetic peptide
KCPYC

SEQ ID NO: 162 - protein, 5 aa, Artificial Sequence
  synthetic peptide
RCPYC

SEQ ID NO: 163 - protein, 5 aa, Artificial Sequence
  synthetic peptide
HCGHC

SEQ ID NO: 164 - protein, 5 aa, Artificial Sequence
  synthetic peptide
KCGHC

SEQ ID NO: 165 - protein, 5 aa, Artificial Sequence
  synthetic peptide
RCGHC

SEQ ID NO: 166 - protein, 5 aa, Artificial Sequence
  synthetic peptide
CRPYC

SEQ ID NO: 167 - protein, 6 aa, Artificial Sequence
  synthetic peptide
KCRPYC

SEQ ID NO: 168 - protein, 7 aa, Artificial Sequence
  synthetic peptide
KHCRPYC

SEQ ID NO: 169 - protein, 6 aa, Artificial Sequence
  synthetic peptide
RCRPYC

SEQ ID NO: 170 - protein, 6 aa, Artificial Sequence
  synthetic peptide
HCRPYC

SEQ ID NO: 171 - protein, 5 aa, Artificial Sequence
  synthetic peptide
CPRYC

SEQ ID NO: 172 - protein, 6 aa, Artificial Sequence
  synthetic peptide
KCPRYC

SEQ ID NO: 173 - protein, 6 aa, Artificial Sequence
  synthetic peptide
RCPRYC

SEQ ID NO: 174 - protein, 6 aa, Artificial Sequence
  synthetic peptide
HCPRYC

SEQ ID NO: 175 - protein, 5 aa, Artificial Sequence
  synthetic peptide
CPYRC

SEQ ID NO: 176 - protein, 6 aa, Artificial Sequence
  synthetic peptide
KCPYRC

SEQ ID NO: 177 - protein, 6 aa, Artificial Sequence
  synthetic peptide
RCPYRC

SEQ ID NO: 178 - protein, 6 aa, Artificial Sequence
  synthetic peptide
HCPYRC

SEQ ID NO: 179 - protein, 5 aa, Artificial Sequence
  synthetic peptide
CKPYC

SEQ ID NO: 180 - protein, 6 aa, Artificial Sequence
  synthetic peptide
KCKPYC

SEQ ID NO: 181 - protein, 6 aa, Artificial Sequence
  synthetic peptide
RCKPYC

SEQ ID NO: 182 - protein, 6 aa, Artificial Sequence
  synthetic peptide
HCKPYC

SEQ ID NO: 183 - protein, 5 aa, Artificial Sequence
  synthetic peptide
CPKYC

SEQ ID NO: 184 - protein, 6 aa, Artificial Sequence
  synthetic peptide
KCPKYC

SEQ ID NO: 185 - protein, 6 aa, Artificial Sequence
  synthetic peptide
RCPKYC

SEQ ID NO: 186 - protein, 6 aa, Artificial Sequence
  synthetic peptide
HCPKYC

SEQ ID NO: 187 - protein, 5 aa, Artificial Sequence
  synthetic peptide
CPYKC

SEQ ID NO: 188 - protein, 6 aa, Artificial Sequence
  synthetic peptide
KCPYKC

SEQ ID NO: 189 - protein, 6 aa, Artificial Sequence
  synthetic peptide
RCPYKC

SEQ ID NO: 190 - protein, 6 aa, Artificial Sequence
  synthetic peptide
HCPYKC

SEQ ID NO: 191 - protein, 4 aa, Artificial Sequence
  synthetic peptide
LAVL

SEQ ID NO: 192 - protein, 4 aa, Artificial Sequence
  synthetic peptide
TVQA

SEQ ID NO: 193 - protein, 4 aa, Artificial Sequence
  synthetic peptide
GAVH

SEQ ID NO: 194 - protein, 4 aa, Artificial Sequence
  synthetic peptide
  PEPTIDE (1)..(1): X can be any amino acid
XAVL

SEQ ID NO: 195 - protein, 4 aa, Artificial Sequence
  synthetic peptide
  PEPTIDE (2)..(2): X can be any amino acid
LXVL

SEQ ID NO: 196 - protein, 4 aa, Artificial Sequence
  synthetic peptide
  PEPTIDE (3)..(3): X can be any amino acid
LAXL

SEQ ID NO: 197 - protein, 4 aa, Artificial Sequence
  synthetic peptide
  PEPTIDE (4)..(4): X can be any amino acid
LAVX

SEQ ID NO: 198 - protein, 4 aa, Artificial Sequence
  synthetic peptide
  PEPTIDE (1)..(1): X can be any amino acid
XVQA

SEQ ID NO: 199 - protein, 4 aa, Artificial Sequence
  synthetic peptide
  PEPTIDE (2)..(2): X can be any amino acid
TXQA

SEQ ID NO: 200 - protein, 4 aa, Artificial Sequence
  synthetic peptide
  PEPTIDE (3)..(3): X can be any amino acid
TVXA

SEQ ID NO: 201 - protein, 4 aa, Artificial Sequence
  synthetic peptide
  PEPTIDE (4)..(4): X can be any amino acid
TVQX

SEQ ID NO: 202 - protein, 4 aa, Artificial Sequence
  synthetic peptide
  PEPTIDE (1)..(1): X can be any amino acid
XAVH

SEQ ID NO: 203 - protein, 4 aa, Artificial Sequence
  synthetic peptide
  PEPTIDE (2)..(2): X can be any amino acid
GXVH

SEQ ID NO: 204 - protein, 4 aa, Artificial Sequence
  synthetic peptide
  PEPTIDE (3)..(3): X is any amino acid
GAXH

SEQ ID NO: 205 - protein, 4 aa, Artificial Sequence
  synthetic peptide
  PEPTIDE (4)..(4): X is any amino acid
GAVX

SEQ ID NO: 206 - protein, 5 aa, Artificial Sequence
  synthetic peptide
PAFPL

SEQ ID NO: 207 - protein, 5 aa, Artificial Sequence
  synthetic peptide
DQGGE

SEQ ID NO: 208 - protein, 5 aa, Artificial Sequence
  synthetic peptide
  PEPTIDE (1)..(1): X can be any amino acid
XAFPL

SEQ ID NO: 209 - protein, 5 aa, Artificial Sequence
  synthetic peptide
  PEPTIDE (2)..(2): X can be any amino acid
PXFPL

SEQ ID NO: 210 - protein, 5 aa, Artificial Sequence
  synthetic peptide
  PEPTIDE (3)..(3): X can be any amino acid
PAXPL

SEQ ID NO: 211 - protein, 5 aa, Artificial Sequence
  synthetic peptide
  PEPTIDE (4)..(4): X can be any amino acid
PAFXL

SEQ ID NO: 212 - protein, 5 aa, Artificial Sequence
  PAFPX
  PEPTIDE (5)..(5): X can be any amino acid
PAFPX

SEQ ID NO: 213 - protein, 5 aa, Artificial Sequence
  synthetic peptide
  PEPTIDE (1)..(1): X can be any amino acid
XQGGE

SEQ ID NO: 214 - protein, 5 aa, Artificial Sequence
  synthetic peptide
  PEPTIDE (2)..(2): X can be any amino acid
DXGGE

SEQ ID NO: 215 - protein, 5 aa, Artificial Sequence
  synthetic peptide
  PEPTIDE (3)..(3): X can be any amino acid
DQXGE

SEQ ID NO: 216 - protein, 5 aa, Artificial Sequence
  synthetic peptide
  PEPTIDE (4)..(4): X can be any amino acid
DQGXE

SEQ ID NO: 217 - protein, 5 aa, Artificial Sequence
  synthetic peptide
  PEPTIDE (5)..(5): X can be any amino acid
DQGGX

SEQ ID NO: 218 - protein, 6 aa, Artificial Sequence
  synthetic peptide
DIADKY

SEQ ID NO: 219 - protein, 6 aa, Artificial Sequence
  synthetic peptide
  PEPTIDE (1)..(1): X can be any amino acid
XIADKY

SEQ ID NO: 220 - protein, 6 aa, Artificial Sequence
  synthetic peptide
  PEPTIDE (2)..(2): X can be any amino acid
DXADKY

SEQ ID NO: 221 - protein, 6 aa, Artificial Sequence
  synthetic peptide
  PEPTIDE (3)..(3): X can be any amino acid
DIXDKY

SEQ ID NO: 222 - protein, 6 aa, Artificial Sequence
  synthetic peptide
  PEPTIDE (4)..(4): X can be any amino acid
DIAXKY

SEQ ID NO: 223 - protein, 6 aa, Artificial Sequence
  synthetic peptide
  PEPTIDE (5)..(5): X can be any amino acid
DIADXY

SEQ ID NO: 224 - protein, 6 aa, Artificial Sequence
  synthetic peptide
  PEPTIDE (6)..(6): X can be any amino acid
DIADKX

SEQ ID NO: 225 - protein, 4 aa, Artificial Sequence
  synthetic peptide
KCHC

SEQ ID NO: 226 - protein, 4 aa, Artificial Sequence
  synthetic peptide
RCKC

SEQ ID NO: 227 - protein, 4 aa, Artificial Sequence
  synthethic peptide
CPYC

SEQ ID NO: 228 - protein, 5 aa, Artificial Sequence
  synthetic peptide
HCPYC

SEQ ID NO: 229 - protein, 5 aa, Artificial Sequence
  synthetic peptide
KCPYC

SEQ ID NO: 230 - protein, 5 aa, Artificial Sequence
  synthetic peptide
RCPYC

SEQ ID NO: 231 - protein, 5 aa, Artificial Sequence
  synthetic peptide
CRPYC

SEQ ID NO: 232 - protein, 5 aa, Artificial Sequence
  synthetic peptide
CPRYC

SEQ ID NO: 233 - protein, 5 aa, Artificial Sequence
  synthetic peptide
CPYRC

SEQ ID NO: 234 - protein, 5 aa, Artificial Sequence
  synthetic peptide
CKPYC

SEQ ID NO: 235 - protein, 5 aa, Artificial Sequence
  synthetic peptide
CPKYC

SEQ ID NO: 236 - protein, 5 aa, Artificial Sequence
  synthetic peptide
CPYKC

SEQ ID NO: 237 - protein, 6 aa, Artificial Sequence
  synthetic peptide
RCRPYC

SEQ ID NO: 238 - protein, 6 aa, Artificial Sequence
  synthetic peptide
RCPRYC

SEQ ID NO: 239 - protein, 6 aa, Artificial Sequence
  synthetic peptide
RCPYRC

SEQ ID NO: 240 - protein, 6 aa, Artificial Sequence
  synthetic peptide
RCKPYC

SEQ ID NO: 241 - protein, 6 aa, Artificial Sequence
  synthetic peptide
RCPKYC

SEQ ID NO: 242 - protein, 6 aa, Artificial Sequence
  synthetic peptide
RCPYKC

SEQ ID NO: 243 - protein, 6 aa, Artificial Sequence
  synthetic peptide
KCRPYC

SEQ ID NO: 244 - protein, 6 aa, Artificial Sequence
  synthetic peptide
KCPRYC

SEQ ID NO: 245 - protein, 6 aa, Artificial Sequence
  synthetic peptide
KCPYRC

SEQ ID NO: 246 - protein, 6 aa, Artificial Sequence
  synthetic peptide
KCKPYC

SEQ ID NO: 247 - protein, 6 aa, Artificial Sequence
  synthetic peptide
KCPKYC

SEQ ID NO: 248 - protein, 6 aa, Artificial Sequence
  synthetic peptide
KCPYKC